(19) United States
(12) Patent Application Publication
(10) Pub. No.: US 2004/0053873 A1
(43) Pub. Date: Mar. 18, 2004

(54) Electroporation methods for introducing bioactive agents into cells

(76) Inventors: Shikha P. Barman, Bedford, MA (US); Mary Lynne Hedley, Lexington, MA (US); Daqing Wang, Bedford, MA (US)

Correspondence Address: FISH & RICHARDSON PC, 225 FRANKLIN ST, BOSTON, MA, 02110, US

(21) Appl. No.: 10/370,131

(22) Filed: Feb. 19, 2003

Related U.S. Application Data
(60) Provisional application No. 60/357,542, filed on Feb. 15, 2002.

(51) Int. Cl. A61K 48/00; C12N 15/87; A61K 38/00

(52) U.S. Cl. 514/44; 435/461; 514/12

(57) ABSTRACT

The invention provides compositions and methods for introducing bioactive agents into cells. Bioactive agents are provided together with a delivery vehicle and a cell is subjected to electroporation, thereby resulting in the introduction of the bioactive agent into the cell.

### CROSS REFERENCE TO RELATED APPLICATIONS

This application claims priority from U.S. Provisional Application No. 60/357,542, filed Feb. 15, 2002, the content of which is incorporated herein by reference in its entirety.

### FIELD OF THE INVENTION

The invention relates generally to methods of introducing bioactive agents into cells.

### BACKGROUND OF THE INVENTION

Various techniques have been used for introducing nucleic acids and other foreign material into cells or tissues of an organism, including biolistic transfer, viral-mediated gene transfer, injection of “naked” DNA (U.S. Pat. No. 5,580,859), delivery via cationic liposomes (U.S. Pat. No. 5,264,618), and delivery via microparticles (U.S. Pat. No. 5,783,567). Additional non-viral methods of gene transfer include lipofection/liposome fusion ((1993) Proc. Nat'l Acad. Sci. 84:7413-7417) and the use of polymers admixed with nucleic acids in solution and delivered to muscle tissue (U.S. Pat. No. 6,040,295).

The use of electric pulses for cell permeabilization has also been used to introduce foreign material such as nucleic acids into cells (Somiari et. al. (2000) Molecular Therapy 2:178-87; Mathiesen (1999) Gene Therapy 6:508-514; U.S. Pat. No. 6,261,281). This process, also termed electropermeabilization, allows efficient cytoplasmic uptake of, for example, large and highly charged polynucleotide molecules. The application of controlled electric pulses to cells opens up “pores” in cell membranes through which polynucleotides and other macromolecules of interest may pass across a concentration gradient into the interior of a cell. Over time, after initial permeabilization, the pores reseal, entrapping molecules that may, in turn exert a biological effect.

### SUMMARY OF THE INVENTION

The invention is based on the discovery that a bioactive agent contained in a delivery vehicle can be efficiently introduced into a cell by electroporation. The methods described herein can result in enhanced and/or prolonged activity of the bioactive agent following its introduction into a cell.

In one aspect, the invention features a method of introducing a bioactive agent into a living cell, including the steps of: contacting a living cell with a delivery vehicle containing a bioactive agent; and applying an electrical field via electroporation to the cell, under conditions and for sufficient time to allow uptake of the bioactive agent into the cell, wherein the delivery vehicle is a microparticle or a hydrogel, and wherein the microparticle is not encapsulated in a liposome.

In another aspect, the invention features a method of introducing a bioactive agent into a living cell, including the steps of: contacting a living cell with a delivery vehicle containing a bioactive agent; and applying an electrical field via electroporation to the cell, under conditions and for sufficient time to allow uptake of the bioactive agent into the cell.

A “bioactive agent” is any substance that has a biological effect on a cell. The term includes, for example, polypeptides (of any length), nucleic acids (of any length), macromolecules, small molecules, carbohydrates, lipids, as well as any type of drug.

“Electroporation” refers to the application of an electric pulse to a cell by an individual that results in permeabilization of the cell membrane. Electroporation does not encompass naturally occurring phenomena. The terms “electroporation” and “electropermeabilization” are used interchangeably. The application of controlled electric pulses to cells is thought to open up “pores” in cell membranes through which bioactive agents may pass across a concentration gradient into the interior of the cell. Over time, the pores reseal, entrapping bioactive agents in a cell, which in turn exert a biological effect (see, e.g., U.S. Pat. Nos. 5,993,434 and 6,096,020). The pores resulting from electroporation are believed to range in size from about 20-120 nm in diameter (Chang et al. (1990) Biophys. J. 1990 58:1-12).

A “delivery vehicle” refers to a composition that facilitates the introduction of a bioactive agent into a cell. A “delivery vehicle” promotes the introduction of the bioactive agent into a cell, resulting in enhanced and/or prolonged activity of the bioactive agent as compared to that resulting when the cell is contacted with the bioactive agent in the absence of the delivery vehicle. Accordingly, “delivery vehicle” does not refer to water or other physiological buffers. A delivery vehicle generally contains the bioactive agent (e.g., encapsulates or embeds the bioactive agent), physically associates with the bioactive agent (e.g., is in an aqueous solution with the bioactive agent), or forms a complex with the bioactive agent (e.g., forms a covalent or non-covalent complex with the bioactive agent). Examples of delivery vehicles include microparticles, microspheres, microcapsules, hydrogels, depots, liposomes, suspensions, colloids, emulsions, dispersions, pellets, implants, pumps, particulates, polymeric networks, immune stimulating complexes (ISCOMs), and microorganisms such as viruses and bacteria.

The bioactive agent used in the methods described herein can be a nucleic acid (e.g., DNA or an RNA molecule such as RNAi or siRNA), a viral DNA, an oligonucleotide or plasmid DNA, or a peptide nucleic acid. The nucleic acid can optionally encode a polypeptide and the methods described herein can result in production of the polypeptide by the cell.

In those embodiments where the bioactive agent is a nucleic acid encoding a polypeptide, the methods can result in detectable expression of the polypeptide produced by the cell for a period of at least 1, 2, 3, 4, 5, 6, 7, 8, 9, 10, 11, 12, 13, 14, 15, 16, 17, 18, 19, 20, or more weeks, or 3, 6, 9 or 12 months, or a year or more. The levels of the polypeptide detected at any of these time points can be, for example, at least 10 pg, 0.1 ng, 1 ng, 10 ng, 100 ng, 1 μg, or more of the polypeptide.

In those embodiments where the bioactive agent is a nucleic acid encoding a polypeptide, the methods can include a step of detecting expression of the polypeptide produced by the cell after a period of at least 1, 2, 3, 4, 5, 6, 7, 8, 9, 10, 11, 12, 13, 14, 15, 16, 17, 18, 19, 20, or more weeks, or 3, 6, 9 or 12 months, or a year or more. Because of the unexpectedly prolonged expression of a nucleic acid that can be achieved by the practice of the methods described herein, an encoded polypeptide can be detected for unexpectedly long periods of time after an initial administration of the nucleic acid. The bioactive agent used in any of the methods described herein can be a polypeptide.

The contacting and applying steps of the methods described herein can be carried out on a cell or population of cells or a tissue or organ in vitro or in vivo. In those embodiments where the steps are carried out in vitro, the cell or population of cells or a tissue or organ can be introduced into an animal following the introduction of the bioactive agent. The methods of delivery encompass the ex vivo methods of treatment.

In one embodiment, the cell or population of cells or a tissue or organ is contained in a living animal, e.g., a human, non-human primate, dog, pig, mouse, or rat, and the methods includes applying an electrode to a tissue, e.g., a muscle tissue, of the animal. In other embodiments, the cell is contained in a living plant. In those embodiments where the bioactive agent is a nucleic acid encoding a polypeptide, the method can result in detectable expression of the polypeptide produced by the cell for a period of at least 1, 2, 3, 4, 5, 6, 7, 8, 9, 10, 11, 12, 13, 14, 15, 16, 17, 18, 19, 20, or more weeks, or 3, 6, 9 or 12 months, or a year or more. The polypeptide can be detected in, for example, serum, bodily fluids (e.g., saliva, sperm, tears, sweat, urine), or a solid tissue of the animal. In those embodiments where the polypeptide is detected in the serum, the levels detected can be, for example, at least 10 pg, 0.1 ng, 1 ng, 10 ng, 100 ng, 1 μg, or more of the polypeptide.

In those embodiments where the bioactive agent is a nucleic acid encoding a polypeptide, the method can include a step of detecting expression of the polypeptide produced by a cell of the animal after a period of at least 1, 2, 3, 4, 5, 6, 7, 8, 9, 10, 11, 12, 13, 14, 15, 16, 17, 18, 19, 20, or more weeks, or 3, 6, 9 or 12 months, or a year or more. Because of the unexpectedly prolonged expression of a nucleic acid that can be achieved by the practice of the methods described herein, an encoded polypeptide can be detected at unexpectedly long periods of time after an initial administration of the nucleic acid to an animal.

The methods described herein can result in the generation of an immune response within the animal directed against the polypeptide. In one example, the immune response is a therapeutic immune response. In another example, the immune response is a prophylactic immune response. Immune response can include, activation of NK cells, macrophages, B-cells, T-cells, antibody production, and interleukin and/or cytokine production.

The methods described herein can include injecting an aqueous solution containing the delivery vehicle and the bioactive agent into a tumor, a tissue, e.g., a muscle tissue, or organ of an animal.

The delivery vehicle used in any of the methods described herein can be a microparticle.

In those embodiments where the delivery vehicle is a microparticle, the microparticle can be comprised of a synthetic polymer. For example, the synthetic polymer can be poly-lactide-co-glycolide. In other examples, the microparticle contains biodegradable linkages containing lactates, glycolates, lactate-co-glycolates, caproates, trimethylene carbonates or combinations thereof.

In those embodiments where the delivery vehicle is a microparticle, the microparticle can be less than 10 μm in diameter. In other embodiments, the microparticle is at least 500 nm, 600 nm, 700 nm, 800 nm, 900 nm, 1 μm, 2 μm, 5 μm or more in diameter. Because of the size range of pores that are thought to be generated in a cell membrane by electroporation, it was unexpected that electroporation would result in enhanced delivery and/or prolonged activity of bioactive agents contained in a microparticle having a diameter of, for example, greater than 1 μm.

In some embodiments where the delivery vehicle is a microparticle, the microparticle does not include a catioinic lipid.

The delivery vehicle used in any of the methods described herein can be in an aqueous solution. In some examples, the aqueous solution contains an excipient. Examples of excipients include cell-lytic peptides, polymers, lipids, adjuvants, and bioavailability enhancers.

The delivery vehicle used in any of the methods described herein can be a hydrogel. For example, the methods include the use of a hydrogel composition as described in WO 02/057424.

The bioactive agents (e.g., nucleic acids or polypeptides) described herein may be used for the preparation of a medicament for use in any of the methods described herein (e.g., methods of delivering a bioactive agent to a subject). A bioactive agent can optionally be formulated as pharmaceutical composition for such uses.

An advantage of the delivery methods of the invention is that they can result in unexpectedly enhanced activity of a bioactive agent. For example, in those embodiments where the bioactive agent is a nucleic acid encoding a polypeptide, the methods can result in enhanced expression of the nucleic acid and thereby result in enhanced production of a polypeptide encoded by the nucleic acid. Alternatively, the methods can result in enhanced uptake of the bioactive agent which results in an increased level of expression. Alternatively, the methods can result in enhanced stability of the bioactive agent which results in an increased level of expression. By “enhanced activity” is meant a level of activity of the bioactive agent that exceeds that detected when the bioactive agent is administered to a cell either with the delivery vehicle (in the absence of electroporation) or by electroporation (in the absence of the delivery vehicle).

An additional advantage of the delivery methods of the invention is that they can result in unexpectedly prolonged activity of a bioactive agent. For example, in those embodiments where the bioactive agent is a nucleic acid encoding a polypeptide, the methods can result in prolonged expression of the nucleic acid and thereby result in prolonged production of the polypeptide encoded by the nucleic acid. By “prolonged activity” is meant the maintenance of the activity of a bioactive agent, e.g., expression of a nucleic acid, at a defined threshold level for a period of time that exceeds the duration of activity at that threshold level when the bioactive agent is administered to a cell either with the delivery vehicle (in the absence of electroporation) or by electroporation (in the absence of the delivery vehicle).

Enhanced and/or prolonged activity of a bioactive agent can optionally be detected indirectly by measuring a surrogate indicator that is the result of an enhanced and/or prolonged activity of the bioactive agent. For example, the bioactive agent can cause a biological response such as activation of an immune response, suppression of an immune response, production of a cytokine, reduction in a substrate level (e.g., if the bioactive agent is an enzyme or a nucleic acid encoding an enzyme), or an increase the level of a product of an enzymatic reaction. Such resulting biological responses can be measured to detect an enhanced and/or prolonged activity of the bioactive agent.

Enhanced and/or prolonged activity of a bioactive agent is advantageous in that it can permit the reduction or elimination of the need for repeated administrations of a bioactive agent, e.g., a polypeptide or a nucleic acid encoding a polypeptide. For example, if a bioactive agent is available to a cell in relatively increased amounts and/or for longer periods of time, then fewer doses of the bioactive agent and/or lower doses of the bioactive agent can be administered to the cell to achieve a desired biological effect.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described below. All publications, patent applications, patents, and other references mentioned herein are incorporated by reference in their entirety. In case of conflict, the present application, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a graph depicting the effect of electroporation (EPT) on serum secreted embryonic alkaline phosphatase (SEAP) levels in C57/BL6 mice administered microparticles containing DNA encoding SEAP. Mice were injected intramuscularly with the formulation in the presence or absence of EPT. Control mice given saline were negative for SEAP expression (<0.3 ng/ml) at all time points tested. Serum samples were tested on day 7 (black bar), day 21 (white bar), day 49 (light gray bar), day 90 (dark gray bar), day 200 (diamond bar), and day 300 (diagonal line bar).

FIG. 2 is a graph depicting the effect of EPT on serum secreted SEAP levels at 7 days in Balb/c mice administered microparticles containing DNA encoding SEAP at doses of 30, 10, and 3 μg, as indicated on the x-axis. Serum SEAP levels were tested on day 7. Data from mice injected with microparticles in the absence of EPT (black bars), microparticles combined with EPT (hatched bars), and saline (white bars) are indicated. P values were calculated using a Student's t test (*p=0.004, 30 μg; **p=0.0009, 10 μg; ***p=0.0004, 3 μg).

FIG. 3 is a graph depicting the effect of EPT on P-gal specific IgG titers in Balb/c mouse serum at 23 days post-injection. Mice were injected with encapsulated β-gal-encoding DNA at doses of 30 and 10 μg. Average titers for each group are indicated on the y-axis and the formulation is indicated on the x-axis. The data is presented as the mean±standard error (SE) of five mice. Titers are shown for groups that received encapsulated β-gal-encoding DNA in the absence of EPT (black bars) and those that received encapsulated DNA with EPT (white bars). P values were calculated using a Student's t test (*p=0.006, 30 μg; **p=0.009, 10 μg).

FIG. 4 is a graph depicting the effect of EPT on the number of IFN-γ spot-forming cells (SFC) elicited from pooled spleens of Balb/c mice (n=5 per group) at 6 weeks post-immunization with microparticle-encapsulated β-gal-encoding DNA. The number of IFN-γ SFC/10⁶ CD3+ T cells is indicated on the y-axis. Groups received encapsulated DNA in the absence of EPT (black bars), encapsulated DNA together with EPT (hatched bars), or saline (white bars).

FIG. 5 is a graph depicting the effect of EPT on serum SEAP levels in mice injected intramuscularly with a GT20 P4-AM/P4-SG polymeric network formulation. Groups received the formulation without EPT (GT20), the formulation with EPT (GT20+ EPT), or saline. Serum SEAP level were measured 7 days post administration.

FIGS. 6A and 6B are graphs depicting in vitro release of CPG oligophosphorothioates (ODN) from PLG microparticles. FIG. 6A depicts percent cumulative ODN released from PLG microparticles as a function of time. FIG. 6B depicts the amount (in μg) of ODN released from PLG microparticles as a function of time.

### DETAILED DESCRIPTION OF THE INVENTION

The invention relates to methods of introducing bioactive agents into cells by using a delivery vehicle combined with electroporation to result in enhanced and/or prolonged activity or availability of the bioactive agents. The enhanced or prolonged activity described herein can occur via many mechanisms, such as protection of the bioactive agents from degradation or sustained release from a delivery vehicle. The methods of the invention can be used for a variety of functions including but not limited to the induction of cell activation, the inhibition of cell activation, the inhibition or promotion of cell division, the induction of cell death, the activation or suppression of the immune system, the regulation of gene expression, the induction of gene expression, or the regulation of protein expression or activity.

Bioactive Agents

As described herein, a bioactive agent can be associated with a delivery vehicle and efficiently introduced into a cell by electroporation. Bioactive agents include polypeptides, small molecules, carbohydrates, lipids, and nucleic acids, as well as other types of macromolecules and drugs.

Enhanced or prolonged activity of bioactive agents may be achieved by the combination of a delivery vehicle and electroporation in many ways including, but not limited to, maximizing delivery or protecting the bioactive agent from degradation. Enhanced activity may also occur by modulating release of the bioactive agent from the delivery vehicle with electroporation. The methods of the invention can be used to deliver nucleic acids in a eukaryotic system (e.g., in a cell, tissue, organ, or in an animal). For example, a nucleic acid such as an RNAi, siRNA, oligonucleotide, cDNA, gene, or gene fragment can be encapsulated in a microparticle and injected into the muscle of an animal. The injection site is then electroporated. When a nucleic acid such as a cDNA, gene, or gene fragment is delivered, expression of the delivered nucleic acid can then be monitored. RNAi, siRNA, and oligonucleotides can be used to reduce gene expression, whereas cDNAs, genes, and gene fragments, are frequently used to induce gene expression. Gene expression can be monitored by mechanisms such as chemiluminescense, ELISA, Western, RT-PCR, fluorescence activated cell sorting (FACS), and immunohistochemistry.

In those embodiments where the bioactive agent is a nucleic acid, the nucleic acid can be RNA, DNA, or PNA (peptide nucleic acid). Examples of nucleic acids that can be used in the methods of the invention include, for example, cDNA, genomic DNA, oligonucleotides, mRNA, RNAi, siRNA, viral DNA, bacterial DNA, plasmid DNA, condensed DNA, and peptide nucleic acids (PNAs).

In those embodiments where the nucleic acid encodes a polypeptide, the nucleic acid can be used in a vector that allows expression of the polypeptide. For example, the nucleic acid can be cloned into an expression vector, i.e., a vector in which the coding sequence is operably linked to expression control sequences. The need for, and identity of, expression control sequences will vary according to the type of cell in which the DNA is to be expressed. Generally, expression control sequences include a transcriptional promoter, enhancer, suitable ribosomal binding sites, translation start site, and sequences that terminate transcription and translation, including polyadenylation and possibly translational control sequences. Suitable expression control sequences can be selected by one of ordinary skill in the art. Nucleic acids encoding a polypeptide as described herein can encode a methionine residue at the amino terminus of the polypeptide. Standard methods can be used by the skilled person to construct expression vectors. See generally, Current Protocols in Molecular Biology, 2001, Wiley Interscience, NY. Vectors useful in this invention include linear nucleic acid fragments or circular DNAs, plasmid vectors, supercoiled DNA, viral vectors, fungal vectors, and bacterial vectors.

A “plasmid” is an autonomous, self-replicating, extrachromosomal, circular DNA. An example of a suitable plasmid vector is the family of pcDNA mammalian expression vectors (Invitrogen), which permit direct and rapid cloning of PCR products.

Preferred viral vectors are those derived from baculovirus, retroviruses, adenovirus, adeno-associated virus, pox viruses, SV40 virus, alpha viruses, or herpes viruses.

Nucleic acids introduced into a cell by the methods of the invention can include nuclear localization signals that promote the translocation of the nucleic acid to the nucleus. For example, a nucleic acid can include a sequence of nucleotides that is bound by a DNA binding protein, such as a transcription factor. In another example, a peptide based nuclear localization signal can be provided with a nucleic acid of the invention, to thereby promote the translocation of the nucleic acid to the nucleus. Examples of useful signals include hnRNPA sequences and the SV40 nuclear localization signal. A nuclear localization peptide sequence can be, for example, mixed with a nucleic acid, conjugated to a nucleic acid, or incorporated in a delivery vehicle such as a liposome. Regulatory elements can be included in the nucleic acid to facilitate expression of the nucleic acid encoding a polypeptide. These elements include sequences for enhancing expression in human or other mammalian cells, e.g., promoters and/or enhancers. For example, a CMV promoter, RSV promoter, T7, SP6, or T3 RNA polymerase promoter, tissue-specific promoter such as a muscle-specific promoter, cell-specific promoter such as an antigen presenting cell (APC)-specific promoter, or inducible promoter is optionally present at the 5′ end of the coding sequence. Examples of inducible promoters include a metallothionine promoter (see, e.g., Testa et al. (1994) Cancer Res. 54:4508) a tetracycline-responsive promoter (see, e.g., Giavazzi et al. (2001) 61:309)

The nucleic acid can also include an RNA stabilization sequence, e.g., an RNA stabilization sequence derived from the Xenopus laevis β-globin gene, 5′ and/or 3′ to the coding sequence; an intron (which can be placed at any location within or adjacent to the coding sequence); a poly(A) addition site; an origin of replication; and one or more genes encoding antibiotic, auxotrophic, or other selectable markers, e.g., a kanamycin resistance gene, enabling the constructs to replicate and be selected in prokaryotic and/or eukaryotic hosts.

The nucleic acid may also contain other transcriptional and translational signals, such as a Kozak sequence, as well as a sequence encoding tag such as FLAG, myc, HA, or His, optionally present at the 3′ or 5′ end of the coding sequence.

In those embodiments where the nucleic acid encodes a polypeptide, the encoded polypeptide can be, for example, a therapeutic polypeptide or a reporter polypeptide. A “therapeutic polypeptide” is a polypeptide that induces a beneficial biological effect on either the cell, organ, or tissue in which it is produced and/or another cell, organ or tissue that it contacts (e.g., a secreted polypeptide that stimulates a cell other than the cell that produces the polypeptide). A “reporter polypeptide” provides a detectable signal that serves as an indicator that the nucleic acid has been expressed in a given cell. Nucleic acids encoding reporter polypeptides can be used to verify gene transfer and are therefore particularly useful in screening assays and as positive controls. Examples of useful reporter polypeptides include secreted embryonic alkaline phosphatase (SEAP; see detailed description in Examples), α-galactosidase, luciferase, and green fluorescent protein (GFP). Examples of therapeutic polypeptides include proteins that stimulate immune responses (as described in detail in subsequent sections), chemokines, enzymes (e.g., glucocerebrosidase or alpha galactosidase), cytokines (e.g., IL-12 or IL-2), growth or differentiation factors (e.g., erythropoietin or GM-CSF), or hormones (e.g., hGH, aMSH, or insulin).

Nucleic acids used in the methods of the invention can also be or contain ribozymes. Ribozymes are fragments of RNA that act as enzymes and perform numerous functions. Over-expression of too much of a particular protein can lead to many diseases including cancer. Rather than attack the proteins after they have been produced, ribozymes attack the source: the mRNA. Ribozymes target specific mRNAs through complementary base pair hybridization. After binding to a target, the enzymatic activity of the ribozyme cleaves the target mRNA thus preventing its translation into protein. By choosing mRNA sequences associated with cancer, for example, ribozymes may inhibit cancer progression. After identification of a particular mRNA implicated in a disease process, ribozymes can be used to decrease the amount of that mRNA. For example, hepatitis C virus (HCV) is a causative agent of chronic viral hepatitis. Targeting a sequence in the viral RNA may cause a decrease in mRNA levels leading to decrease in HCV. Using the techniques of modem molecular biology ribozymes can be designed, synthesized and delivered to a mammal or eukaryotic cell. Chemical modifications allow ribozymes to be stable and active in serum for several days.

A nucleic acid (e.g., RNAi or siRNA) can be introduced into a cell according to the methods of the invention for the purpose of RNA interference. RNA interference causes gene-specific silencing and works via double-stranded RNA (dsRNA). Separate injection of antisense mRNA and sense mRNA into C. elegans inhibits gene expression in a sequence-specific manner because the dsRNA targets complementary mRNAs for degradation (Fire et al. (1998) Nature 19:806-11).

Recently it was discovered that interfering RNA duplexes 21 nucleotides in length can mediate gene silencing in cultured mammalian cells without inducing apoptosis (Elbashir et al. (2001) Nature 411:494-98). While long enough to initiate gene specific silencing, the 21-nucleotide duplexes are not long enough to elicit a non-sequence specific interferon response. Oligonucleotides can be introduced into a cell according to methods of the invention. Oligonucleotides can be antisense compounds that target a particular disease-associated or undesirable RNA or mRNA (e.g., a mRNA encoding an oncoprotein). Antisense oligonucleotides are complementary to the target RNA and upon interaction with it will prevent RNA translation or promote RNA degradation. Oligonucleotides can also be immunostimulating compounds as described in U.S. Pat. No. 6,239,116.

Delivery of Bioactive Agents to Modulate an Immune Response

Bioactive agents can be introduced into a cell according to the methods of the invention to modulate (e.g., increase or decrease) an immune response.

Examples of bioactive agents include, but are not limited to, antigens or nucleic acids encoding antigens that can be used to vaccinate against viral, bacterial, protozoan, or fungal infections such as influenzae, respiratory syncytial, parainfluenza viruses, Hemophilus influenza, Bordetella pertussis, Neisseria gonorrhoeae, Streptococcus pneumoniae, anthrax, smallpox, human immunodeficiency virus, human papilloma virus, herpes simplex virus, hepatitis B virus, hepatitis C virus, Plasmodium falciparum, and other infections caused by pathogenic microorganisms. Additional examples of bioactive agents include antigens or nucleic acids encoding antigens to vaccinate against diseases caused by macroorganisms such as helminthic pathogens as well as antigens or nucleic acids encoding antigens to vaccinate against allergies. Additional examples of bioactive agents include immunomodulators (e.g., immunostimulatory agents), nutrients, drugs, peptides, lymphokines, monokines, and cytokines.

Examples of useful immunostimulatory agents include: cytokines such as IL-12, GM-CSF, IL-2, or IFN-gamma; lipopolysaccharide (LPS); monophosphoryl lipid A; QS21; CpG-containing oligonucleotides, e.g., of 18-30 nucleotides in length; and bacterial carbohydrates, lipids or polypeptides such as a bacteriotoxin. Examples of CpG-containing oligonucleotides are described in U.S. Pat. No. 6,239,116.

A nucleic acid as described herein can be an immunostimulatory agent (e.g., a CpG-containing oligonucleotide) or encode an immunostimulatory agent (e.g., a cytokine). A nucleic acid encoding a polypeptide and an immunostimulatory agent can optionally be included in a single vector, e.g., a two promoter vector or IRES vector or any other vector that is capable of expressing multiple genes from a single cistron (e.g., Gaken et al. (2000) Gene Therapy 7:1979-1985). Alternatively, a nucleic acid can encode a polypeptide or portion thereof fused in frame to an immunostimulatory agent. Methods of creating such fusion proteins are well known in the art and are described in, for example, WO 95/05849.

Nucleic acids used in the methods of the invention can contain unmethylated CpG sequences that are present in bacterial DNA but are under represented and methylated in vertebrate DNA. Immune activation in response to CpG DNA may have evolved as one component of the innate immune defense mechanism to microbial molecules. Plasmid vectors containing these short immunostimulatory sequences have been shown to modify immune responses more readily than those without these sequences (Sato et al. (1996) Science 273: 352-354).

Nucleic acids or oligonucleotides containing CpG sequences may enter immune system cells, interact with proteins in the cytoplasm and turn on cell signaling events that activate certain genes. Through this pathway certain CpG molecules activate genes that affect the body's innate immunity, restoring hematopoiesis (the generation of blood cells) and activating broad-spectrum, non-specific therapeutic and prophylactic responses to pathogens or cancer cells. Other CpG sequences activate the body's acquired immunity, stimulating a targeted immune response to specific infectious disease or cancer antigens. Finally, CpG-based products may also prevent allergic or asthmatic symptoms by “rebalancing” hypersensitive immune reactions into more normal immune responses.

Oligonucleotides may also be used in the methods of the invention as adjuvants. Immunostimulatory oligonucleotides have been shown to induce Th1 in experimental systems (Carson and Raz, J. Exp. Med. (1997) 186:1621-22). Oligonucleotides have been shown to generate potent immune responses. Oligodeoxynucleotides, particularly those with CG motifs, can turn a poor antigen into a powerful one. Tetanus toxoid, for example, administered to rats through the gastric mucosa, failed to elicit an immune reaction. When combined with an oligonucleotide adjuvant, however, it rivaled that of subcutaneous injection (Eastcott et al. (2001) Vaccine 19:1636-42). Additionally, anti-sense oligonucleotides may be used alone or combined with chemotherapy as anti-tumor agents.

Nucleic acids delivered by the methods of the invention can be passively targeted to macrophages and other types of professional APC and phagocytic cells, as they represent a means for modulating immune function. Macrophages, monocytes, and dendritic cells serve as professional APCs, expressing both MHC class I and class II molecules. In addition, the mitogenic effect of DNA can be used to stimulate non-specific immune responses mediated by B, T, NK, and other cells. Delivery of an expression vector encoding a foreign antigen which contains peptides that bind to an MHC class I or class II molecule will induce a host T cell response against the antigen, thereby conferring host immunity.

In those embodiments, where the bioactive agent is a nucleic acid encoding a blocking peptide (see, e.g., WO 94/04171) or an altered peptide ligand that binds to an MHC class II molecule involved in autoimmunity, presentation of the autoimmune disease-associated self peptide by the class II molecule is downregulated or prevented, and the symptoms of the autoimmune disease alleviated.

In another example, an MHC binding peptide that is identical or almost identical to an autoimmunity-inducing peptide can affect T cell function by tolerizing or anergizing the T cell. Alternatively, the peptide could be designed to modulate T cell function by altering cytokine secretion profiles (e.g., following recognition of the MHC/peptide complex). Peptides recognized by T cells can induce secretion of cytokines that cause B cells to produce antibodies of a particular class, induce inflammation, and further promote host T cell responses.

Induction of immune responses, e.g., specific antibody responses to peptides or proteins, can require several factors. It is this multifactorial nature that provides impetus for attempts to manipulate immune related cells on multiple fronts, using the delivery methods of the invention. For example, compositions can be prepared and delivered into a cell which carry both DNA and polypeptides within each composition.

Class I/II MHC Restricted T Cell Responses

Antigen presenting cells (APC) present T cell epitopes, small peptide fragments, in the context of class I and II MHC molecules to immature T cells to activate a T cell response, and more specifically a cytotoxic T cell (CTL) or T helper (T_{H}) response. The peptide fragments are known as T cell epitopes. To fully activate CTL and T_{H}, factors in addition to the antigenic peptide are useful. These include certain cytokines such as interleukin-2 (IL-2), IL-12, IL-4 and gamma interferon (γ-IFN). Any factor that promotes the migration, activation, or differentiation of antigen presenting cells or can enhance the development of a T cell response can be provided together in nucleic acid or protein form with nucleic acid encoding the antigen or T cell epitopes.

Nucleic acids useful for activating a T cell response can encode an entire antigen, fragments of an antigen, or several regions of an antigen that each contain one or more T cell epitopes. In addition, individual T cell epitopes can be encoded in a tandem array. A polypeptide have optionally two or more antigenic peptides, wherein the antigenic regions do not overlap. Such tandem arrays of peptides may include two, three, four or more peptides (e.g., up to ten or twenty or more) which can be the same or different. Such tandemly arranged peptides can be interspersed with overlapping peptides. For example, a nucleic acid used in the methods described herein can encode any of the polyepitope polypeptides (e.g., HPV polyepitope polypeptides) described in WO 01/19408.

Antibody Responses

Elimination of certain infectious agents from the host may require both antibody and T cell responses. For example, when the influenza virus enters a host, antibodies can often prevent it from infecting host cells. However, if cells are infected, then a T cell response is required to eliminate the infected cells and to prevent the continued production of virus within the host.

Many antibody responses are directed against conformational determinants and thus require the presence of a protein or a protein fragment containing such a determinant. However, it is known that peptides can also elicit antibody responses when administered with an adjuvant. In contrast, T cell epitopes are usually linear peptide determinants, typically 7-25 residues in length. Thus, when there is a need to induce both a T cell and an antibody response, a delivery vehicle can include an antigenic protein, a nucleic acid encoding an antigenic protein, or both an antigenic protein and a DNA encoding a T cell epitope.

Immunosuppression

Certain immune responses lead to allergy and autoimmunity, and so can be deleterious to the host. In these instances, there is a need to inactivate tissue-damaging immune cells. Immunosuppression can be achieved with microparticles bearing DNA that encodes epitopes that down-regulate T helper (T_{H}) cells or cytotoxic T cells (CTLs), e.g., blocking peptides and tolerizing peptides, altered peptide ligands. Additionally, immunosuppression can be achieved with microparticles bearing DNA encoding certain cytokines, chemokines or other polypeptides (e.g. TGF-β, αMSH, or peptides with αMSH like activity). In these microparticles, the effect of the immunosuppressive DNA could be amplified by including certain proteins in the carrier microparticles with the DNA. A list of such proteins includes antibodies, receptors, transcription factors, and the interleukins.

For example, antibodies to stimulatory cytokines or homing proteins, such as integrins or intercellular adhesion molecules (ICAMs), can increase the efficacy of the immunosuppressive DNA epitope. These proteins serve to inhibit the responses of already-activated T cells, while the DNA further prevents activation of nascent T cells. Induction of T cell regulatory responses can be influenced by the cytokine milieu present when the T cell receptor (TCR) is engaged. Cytokines such as IL-4, IL-10, and IL-6 promote T_{H}2 differentiation in response to the DNA-encoded epitope. T_{H}2 responses can inhibit the activity of T_{H}1 cells and the corresponding deleterious responses which result in the pathologies of rheumatoid arthritis, multiple sclerosis and juvenile diabetes.

Inclusion of proteins comprising soluble forms of costimulatory molecules (e.g., CD-40, gp-39, B7-1, and B7-2), or molecules involved in apoptosis (e.g., Fas, FasL, Bc12, caspase, bax, TNFα, or TNFα receptor) is another way to inhibit activation of particular T cell and/or B cells responses. For example, B7-1 is involved in the activation of T_{H}1 cells, and B7-2 activates T_{H}2 cells. Depending on the response that is required, one or the other of these proteins could be included in the microparticle with the DNA, or could be supplied in separate microparticles mixed with the DNA-containing microparticles. Oligonucleotides can be used in the methods of the invention, for example, to treat asthma. Oligodeoxynucleotides (ODNs), which contain unmethylated motifs centered on CG dinucleotides potently induce Th1 cytokines and suppress Th2 cytokines, and can prevent manifestations of asthma in animal models. These agents have the potential to reverse Th2-type responses to allergens and thus restore balance to the immune system (Hussain and Klin (2001) Curr Opin Investig Drugs 2:914-18).

The methods of the invention can be used to deliver a medicament for the treatment of, for example, cancer, autoimmune diseases, infectious disease, inflammatory disease, or any other condition treatable with a particular defined bioactive agent. Examples of useful medicaments include the polypeptides and nucleic acids described in U.S. Pat. No. 6,013,258, U.S. Pat. No. 6,183,746, WO 01/19408, WO 02/006316, and WO 02/42325 (describing alpha-MSH-containing compositions and compositions useful for generating immune responses against human paplilloma virus proteins and CYP1B1 proteins).

Delivery Vehicles

Bioactive agents can be associated with a delivery vehicle and introduced into a cell by electroporation. Examples of delivery vehicles include microparticles, hydrogels, depots, liposomes, suspensions, colloids, dispersions, pellets, implants, pumps, particulates, polymers, detergents, pluronics, polymeric networks, immune stimulating complexes (ISCOMs), and microorganisms such as viruses and bacteria

Microparticles

Microparticles, including those described in U.S. Pat. No. 5,783,567, WO 00/53161, and WO 01/93835, can be used as vehicles for delivering bioactive agents such as DNA, RNA, or polypeptides into a cell. “Microparticles” include microspheres and microcapsules, e.g., hollow spheres, as well as nanospheres and nanoparticles.

Microparticles can be used to deliver bioactive agents as described herein, optionally with immunostimulatory agents, to a cell, e.g., a cell of an individual. Microparticles contain macromolecules embedded in a polymeric matrix or enclosed in a shell of polymer. Microparticles can maintain the integrity of the macromolecule, e.g., by maintaining the enclosed nucleic acid in a nondegraded state. Microparticles can also be used for pulsed delivery of the macromolecule, (e.g. nucleic acid, DNA, RNA, oligonucleotides, peptides, proteins, lipids) and for delivery at a specific site (e.g., tissue or organ such as muscle) or to a specific cell or target cell population such as phagocytic cells, macrophages, monocytes, or dendritic cells. Microparticle formulations can also be used to activate relevant cell populations such as neutrophils, macrophages, monocytes or dendritic cells.

The polymeric matrix can be a biodegradable co-polymer such as poly-lactic-co-glycolic acid, starch, gelatin, or chitin.

Microparticles may also be formulated as described by Mathiowitz et al. (WO 95/24929) and U.S. Pat. Nos. 5,817,343, 5,922,253, and 6,475,779, herein incorporated by reference.

Polymeric material can be obtained from commercial sources or can be prepared by known methods. For example, polymers of lactic and glycolic acid can be generated as described in U.S. Pat. No. 4,293,539 or purchased from Aldrich.

Alternatively, or in addition, the polymeric matrix can include polylactide, polyglycolide, poly(lactide-co-glycolide), polyanhydride, polyorthoester, polycaprolactone, polyphosphazene, proteinaceous polymer, polypeptide, polyester, or naturally occurring polymers such as alginate, chitosan, and gelatin.

Preferred controlled release substances which are useful in the methods of the invention include the polyanhydrides, co-polymers of lactic acid and glycolic acid wherein the weight ratio of lactic acid to glycolic acid is no more than 4:1, and polyorthoesters containing a degradation-enhancing catalyst, such as an anhydride, e.g., 1% maleic anhydride. Since polylactic acid can take at least one year to degrade in vivo, this polymer should be utilized by itself only in circumstances where extended degradation is desirable.

Association of Nucleic Acid and Polymeric Particles

Polymeric particles containing nucleic acids can be made using a double emulsion technique. First, the polymer is dissolved in an organic solvent. A preferred polymer is polylactic-co-glycolic acid (PLGA), with a lactic/glycolic acid weight ratio of 65:35, 50:50, or 75:25. Next, a sample of nucleic acid suspended in aqueous solution is added to the polymer solution and the two solutions are mixed to form a first emulsion. The solutions can be mixed by vortexing, microfluidization, shaking, sonication, or homogenization. Most preferable is any method by which the nucleic acid receives the least amount of damage in the form of nicking, shearing, or degradation, while still allowing the formation of an appropriate emulsion. For example, acceptable results can be obtained with a Vibra-cell model VC-250 sonicator with a ⅛″ microtip probe, at setting #3, or by controlling the pressure in the microfluidizer, or by using an SL2T Silverson Homogenizer with a ⅝″ tip at 10K.

During this process, water droplets (containing the nucleic acid) form within the organic solvent. If desired, one can isolate a small amount of the nucleic acid at this point in order to assess integrity, e.g., by gel electrophoresis, capillary gel electrophoresis, HPLC.

Alcohol precipitation or further purification of the nucleic acid prior to suspension in the aqueous solution can improve encapsulation efficiency. Precipitation with ethanol has resulted in up to a 147% increase in incorporated DNA and precipitation with isopropanol has increased incorporation by up to 170%.

The nature of the aqueous solution can affect the yield of supercoiled DNA. For example, the presence of detergents such as polymyxin B, which are often used to remove endotoxins during the preparation and purification of DNA samples, can lead to a decrease in DNA encapsulation efficiency. It may be necessary to balance the negative effects on encapsulation efficiency with the positive effects on supercoiling, especially when detergents, surfactants, and/or stabilizers are used during encapsulation. Furthermore, addition of buffer solutions containing either tris (hydroxymethyl) aminomethane (TRIS), ethylenediaminetetraacetic acid (EDTA), or a combination of TRIS and EDTA (TE) resulted in stabilization of supercoiled plasmid DNA, according to analysis by gel electrophoresis. pH effects are also observed. Other stabilizing compounds, such as dextran sulfate, dextrose, dextran, CTAB, polyvinyl alcohol, and sucrose, were also found to enhance the stability and degree of supercoiling of the DNA, either alone or in combination with the TE buffer. Combinations of stabilizers can be used to increase the amount of supercoiled DNA. Stabilizers such as charged lipids (e.g., CTAB), cationic peptides, or dendrimers (J. Controlled Release (1996) 39:357) can also be used. Certain of these can condense or precipitate the DNA. Moreover, stabilizers can have an effect on the physical nature of the particles formed during the encapsulation procedure. For example, the presence of sugars or surfactants during the encapsulation procedure can generate porous particles with porous interior or exterior structures, allowing for a more rapid exit of a drug from the particle. The stabilizers can act to stabilize the bioactive agent, nucleic acid, emulsion, or particles. The stabilizers can act at any time during the preparation of the microspheres: during emulsification, encapsulation or lyophilization, or both, for example.

The first emulsion is then added to an organic solution, allowing formation of microparticles. The solution can be comprised of, for example, methylene chloride, ethyl acetate, acetone, polyvinyl pyrrolidone (PVP) and preferably contains polyvinyl alcohol (PVA). Most preferably, the solution has a 1:100 to 8:100 ratio of the weight of PVA to the volume of the solution. The first emulsion is generally added to the organic solution with stirring in a homogenizer (e.g., a Silverson Model L4RT homogenizer (⅝″ probe) set at 7000 RPM for about 12 seconds) or a microfluidizer.

This process forms a second emulsion which can be subsequently added to another organic solution with stirring (e.g., in a homogenizer, microfluidizer, or on a stir plate). Subsequent stirring causes the first organic solvent (e.g., dichloromethane) to be released and the microspheres to become hardened. Heat, vacuum, or dilution can in addition be used to accelerate evaporation of the solvent. Slow release of the organic solvent (e.g., at room temperature) can result in “spongy” particles, while fast release (e.g., at elevated temperature) results in hollow-core microparticles. The latter solution can be, for example, 0.05% w/v PVA. If sugar or other compounds are added to the DNA, an equal concentration of the compound can be added to the third or fourth solution to equalize osmolarity, effectively decreasing the loss of nucleic acid from the microsphere during the hardening process. The resultant microparticles are washed several times with water to remove the organic compounds. Particles can be passed through sizing screens to selectively remove those larger than the desired size. If the size of the microparticles is not crucial, one can dispense with the sizing step. After washing, the particles can either be used immediately, frozen for later use, or be lyophilized for storage.

Characterization of Microparticles

The size distribution of the microparticles prepared by the methods described herein can be determined with a COULTER™ counter or particle sizer. These instruments provide a size distribution profile and statistical analysis of the particles. Alternatively, the average size of the particles can be determined by visualization under a microscope fitted with a sizing slide or eyepiece.

If desired, the nucleic acid can be extracted from the microparticles for analysis by the following procedure. Microparticles are dissolved in an organic solvent such as chloroform or methylene chloride in the presence of an aqueous solution. The polymer stays in the organic phase, while the nucleic acid goes to the aqueous phase. The interface between the phases can be made more distinct by centrifugation. Isolation of the aqueous phase allows recovery of the nucleic acid. The nucleic acid is retrieved from the aqueous phase by precipitation with salt and ethanol in accordance with standard methods, or the supernatant can be concentrated by drying. To test for concentration, the extracted nucleic acid can be analyzed by UV spectrophotometry, HPLC, or capillary gel electrophoresis. To test for degradation, the extracted nucleic acid can be analyzed by HPLC, capillary gel electrophoresis or agarose gel electrophoresis.

Lipid-Containing Microparticles

The microparticles described herein can also include one or more types of lipids. The inclusion of a lipid in a microparticle can increase the stability of the nucleic acid in the microparticle, e.g., by maintaining a covalently closed double-stranded DNA molecule in a supercoiled state. In addition, the presence of a lipid in the particle can modulate, i.e., increase or decrease, the rate at which a drug or nucleic acid is released from the microparticle. Inclusion of charged lipids may also increase the efficiency of electroporation, since the presence of a charge may facilitate microparticle movement across the electric field.

Addition of a lipid to the microparticle can in certain cases increase the efficiency of encapsulation of the nucleic acid or increase the loading of the nucleic acid within microparticles. For example, the encapsulation efficiency may be improved because the presence of the lipid reduces the surface tension between the inner aqueous phase and the organic phase. Reduction of the surface tension is thought to create an environment more favorable for the nucleic acid, and therefore to increase its retention within the microsphere. A reduction in surface tension also allows for the primary emulsion to be formed with less manipulation, which minimizes shearing of the nucleic acid and increases encapsulation efficiency. It is also possible that the presence of lipid in the microparticle may enhance the stability of the microparticle/nucleic acid formulation, and may increase the hydrophobic nature of the microparticles, thereby increasing uptake by phagocytic cells. The lipids can be cationic, anionic, or zwitterionic, or may carry no charged groups, such as nonpolar glycerides. The lipids preferably are not present as liposomes that encapsulate (i.e., surround) the microparticles. The lipids may optionally form micelles. Examples of lipids that can be used in the microparticles include acids (such as carboxylic acids), bases (such as amines), phosphatidylethanolamine, phosphatidyl glycerol, phosphatidyl serine, phosphatidyl inositol, phosphatidylcholine, phosphatidic acid, containing one or more of the following groups: propinoyl (trianoic), butyroyl (tetranoic), valeroyl (pentanoic), caproyl (hexanoic), heptanoyl (heptanoic), caproyl (decanoic), undecanoyl (undecanoic), lauroyl (dodecanoic) tridecanoyl (tridecanoic), myristoyl (tetradecanoic), pentadecanoyl (pentadecanoic), palmitoyl (hexadecanoic), phytanoyl (3,7,11,15-tetramethylhexadecanoic), heptadecanoyl (heptadecanoic), stearoyl (octadecanoic), bromostearoyl(dibromostearoic), nonadecanoyl (nonadecanoic), arachidoyl (eicosanoic), heneicosanoyl (heneicosanoic), behenoyl (docosanoic), tricosanoyl (tricosanoic), lignoceroyl (tetracosanoic), myristoleoyl (9-cis-tetradecanoic), myristelaidoyl (9-trans-tetradecanoic), palmitoleoyl (9-cis-hexadecanoic), palmitelaidoyl (9-trans-hexadecenoic), petroselinoyl (6-cis-octadecenoic), oleoyl (9-cis-octadecenoic), elaidoyl (9-trans-octadecenoic), linoleoyl (9-cis-12-cis-octadecadienoic), linolenoyl (9-cis-12-cis-15-cis octadecadoenoic), eicosenoyl (11-cis-eicosenoic), arachidonoyl (5,8,11,14 (all cis) eicosatetraenoic), erucoyl (13-cis-docsenoic), and nervonoyl (15-cis-tetraosenoic).

Other suitable lipids include cetyltrimethyl ammonium, which is available as cetyltrimethyl ammonium bromide (“CTAB”).

More than one lipid can be used to make a lipid-containing microparticle. Suitable commercially available lipid preparations include lecithin, OVOTHIN 160™, and EPIKURON 135F™ lipid suspensions, all of which are available from Lucas Meyer, Inc., Decatur, Ill.

The lipid may also be isolated from an organism, e.g., a mycobacterium. The lipid is preferably a CD 1-restricted lipid, such as the lipids described in Pamer, Trend Microbiol. 7:13, 1999; Braud, Curr Opin. Immunol. 11:100, 1999; Jackman, Crit. Rev. Immunol. 19:49, 1999; and Prigozy, Trends Microbiol. 6:454, 1998.

In addition to the lipids incorporated into the microparticles, the microparticles can be suspended in a lipid (or lipid suspension) to improve delivery and to improve dispersion following delivery.

The relative increase or decrease in release observed will depend in part on the type of lipid or lipids used in the microparticle. Examples of lipids that increase the release of nucleic acid from microparticles include CTAB and the lecithin and OVOTHIN™ lipid preparations (see, e.g., WO 00/53161).

The chemical nature of the lipid can affect its spatial relationship with the nucleic acid in the particle. If the lipid is cationic, it may interact directly with the nucleic acid. If the lipid is not charged, it may be interspersed within the microparticle. The lipid may also be in hollow centers of microcapsules or vacuoles of microspheres.

The lipid-containing microparticles may also include the stabilizers described above. The inclusion of a lipid in a microparticle along with a stabilizer such as sucrose can provide a synergistic increase in the release of nucleic acids within the microparticle.

Lipid-containing microparticles can be prepared by adding a lipid to either the organic solvent containing the polymer, to the aqueous solution containing the DNA solution, or to the third solution used to make the second emulsion, as described above. The solubility properties of a particular lipid in an organic or aqueous solvent will determine which solvent is used.

Some lipids or lipid suspensions can be added to either the organic solvent or aqueous solution. However, the release properties of the resulting microparticles can differ. For example, microparticles prepared by adding a lecithin lipid suspension to the aqueous nucleic acid-containing solution release amounts similar to or less than the amount released by microparticles prepared without lipids. In contrast, addition of the lecithin lipid suspension to the organic solvent produces microparticles which release more nucleic acid.

Microparticles may in addition be resuspended in a lipid-containing solution to facilitate resuspension and dispersion of the microparticles.

In addition to the lipid-containing microparticles described herein, microparticles may also be made using other macromolecules such as chitin, gelatin, or alginate, or various combinations of these macromolecules and lipids. These microparticles made with these other macromolecules may in addition include the above-described stabilizing agents.

Reconstitution of Microparticles in Polymers

Microparticles with or without lipids can be delivered in saline or incorporated in other polymers. For example, microparticles such as poly(lactide-co-glycolide) can be incorporated in aqueous solutions of non-ionic polymers such as poly(ethylene oxide) (PEO)(BASF, Inc.), poly(ethylene oxide)-co-(propylene oxide)-poly(ethylene oxide)(PEO-PPO-PEO) (BASF, Inc.), poly(propylene oxide)-co-poly(ethylene oxide)-co-poly(propylene oxide) (PPO-PEO-PPO)(BASF, Inc.), cellulose acetate (Sigma), carboxymethyl cellulose (CMC, Sigma, Inc.), poly(vinyl alcohol) and poly(vinyl pyrrolidinone).

In another example, microparticles such as poly(lactide-co-glycolide) can be incorporated in aqueous solutions of charged polymers such as poly(amino acids)((poly(lysine), poly(arginine), etc.), poly(amidoamine) (PAMAM)(Dendritech, Inc.), poly(ethyleneimine)(PEI)(Sigma, Inc.), poly(aspartic acid)(Sigma, Inc.), poly(glutamic acid)(Sigma, Inc.), poly(acrylic acid)(Sigma, Inc.), chitosan (Pronova, Inc.), hyaluronic acid (Genzyme), chrondoitin sulfate, heparin, heparan sulfate (Sigma).

In another embodiment, microparticles can be incorporated into temperature sensitive polymers or viscosity-increasing polymers such as Pluronics® (BASF), poly(vinyl caprolactam)(Sigma), poly(n-propyl isoacrylamide) and derivatized PEO-PPO-PEO polymers such as Pluronic® lactates/glycolate/caproates/trimethylene carbonates), wherein the microparticles are reconstituted in a cold solution of the polymers and injected or applied as a low viscosity formulation. The formulation increases in viscosity post application to tissue at body temperature (37° C.). This allows the microparticle formulation to form a non-chemically crosslinked gel in place, useful in topical delivery applications.

In another embodiment, microparticles that are coated with charged molecules such as CTAB (Cetriammonium Bromide) (Sigma, Inc.), Sodium lauryl sulfate (SLS)(Sigma, Inc.), DOTAP (dioleyl triammonium phosphate) (Sigma, Inc.) are reconstituted in aqueous solutions are injected pre-electroporation.

In another example, nucleic acid-containing microparticles contain conducting compounds such as cinnamic acid (Sigma, Inc.), azocinnamates, etc. These types of microparticles can respond to electrical pulses and be driven into cells with electroporation.

In another example, cell-permeation enhancers such as cell lytic peptides (e.g. magainin (Sigma, Inc.), cecropin (Sigma, Inc.), streptolysin (Sigma, Inc.), listeriolysin (Sigma, Inc.)) are co-encapsulated with nucleic acids within microparticles. Electroporation is applied post-injection of these microparticles. Co-encapsulation of cell-permeation enhancers may enhance further the cellular nucleic acid uptake. Conversely, the nucleic acid-containing microparticles can be reconstituted in a aqueous solution containing cell permeation enhancers. These may be comprised of peptides such as Magainin, Cecropin, etc., polymeric or small molecule surfactants such as poly(ethylene oxide) (BASF), pluronics® (BASF), sodium decyl sulfate (Sigma, Inc.). In another example, microparticles can be reconstituted in bioavailability enhancers such as Vitamin E, VitaminE-TPGS (pegylated Vitamin E) (Eastman Chemical, Inc.). The use of these enhancers combined with electroporation can enhance cellular uptake of DNA.

ISCOMS

ISCOMs are negatively charged, cage-like structures of 30-40 nm in size formed spontaneously on mixing cholesterol and Quil A (saponin), or saponin alone. Any of the bioactive agents described herein can be introduced into a cell by an ISCOM. Protective immunity has been generated in a variety of experimental models of infection, including toxoplasmosis and Epstein-Barr virus-induced tumors, using ISCOMS as the delivery vehicle for antigens (Mowat et al. (1991) Immunology Today 12:383-385).

In those embodiments where the bioactive agent is a nucleic acid, it is expected that a dosage of approximately 1 to 200 μg of DNA would be administered per kg of body weight per dose. Where the patient is an adult human, vaccination regimens can include, e.g., intramuscular, intranasal, intradermal, subdermal, intraorgan (e.g. liver, kidney, brain) or intrarectal, administrations of 10-1000 μg of DNA when delivered in a microparticle or other delivery vehicle, repeated 3-6 times. Of course, as is well known in the medical arts, dosage for any given patient depends upon many factors, including the patient's size, body surface area, age, sex, and general health; the time and route of administration; the particular compound to be administered; and other drugs being administered concurrently. Determination of optimal dosage is well within the abilities of a pharmacologist of ordinary skill.

Electroporation

Bioactive agents contained in a delivery vehicle are introduced in a cell by electroporation. Electroporation has been used for delivery of a wide variety of therapeutic compositions such as antithrombotic and anticoagulant agents (see, e.g., U.S. Pat. No. 5,944,710), pharmacological compounds (see, e.g., U.S. Pat. No. 5,439,440), and chemotherapeutic agents (see, e.g., U.S. Pat. No. 6,055,453). The technique has been used successfully in several mammalian species (e.g., humans, pigs, chimps, dogs, mice, and rats) to deliver bioactive agents (see, e.g., Tozen et al. (2001) Anticancer Res. 4A:2483-88). Electroporation has been used to deliver foreign DNA into eukaryotic cells (Somiari et al. (2000) Mol. Ther. 2:178-87; Mathiesen Gene Therapy (1999) 6:508-14). Electroporation employs controlled electric pulses to deliver bioactive agents to the cytoplasm of cells. The technique has been shown to be useful in the area of gene therapy (Jaroszeski et al. (1999) Adv Drug Deliv Rev 35:131-137), drug delivery to treat cancer (Heller et al. (1997) Adv. Drug Deliv. Rev. 26:185-97), and antibody delivery to study viruses inside the cell, cancer cells, signal transduction, genetics, metabolism, and other structures and mechanisms.(Baron et al. (2000) J. Immunol. Methods 242:115-26).

Electroporation can be applied to virtually any cell, either in vitro or in vivo (e.g., a skin or muscle cell). In vitro methods may include electroporating cells in culture with a therapeutic bioactive agent and subsequently delivering the cells to a subject in need of the bioactive agent. Apparatuses for electroporation and methods of electroporating cells are well known and are described in, for example, U.S. Pat. Nos. 5,702,359 and 6,014,584. Selecting the appropriate apparatus for and parameters of electroporation can be accomplished by a skilled artisan using the techniques described herein.

Uses

The methods of the invention allow for the introduction of a bioactive agent into a cell. Methods of introducing a bioactive agent into a cell have a wide variety of applications in the biological and medical sciences, including but not limited to those described in detail below. One particularly well known and useful application is the introduction of a nucleic acid into a cell, resulting in the production of a polypeptide encoded by the nucleic acid. This technique is of fundamental importance in both basic research as well as in therapeutic applications.

Bioactive agents (e.g., nucleic acids, peptides, proteins, small molecules, carbohydrates, or lipids) introduced into a cell by methods of the invention can be used as immunogens in individuals known to have various types of cell proliferative disorders, such as lymphoproliferative disorders or cancer, individuals suspected of having various types of cancer, or individuals susceptible to various types of cancer (e.g., individuals having genetic and/or hereditary indicia of cancer susceptibility, e.g., mutations in the BRCA1 gene). Other suitable individuals include those displaying symptoms of, or likely to develop, cancer-associated conditions. The bioactive agents can be used, prophylactically or therapeutically, to prevent or treat conditions associated with several different cell proliferative disorders or cancers, e.g., cancers of the bladder, breast, colon, connective tissue, lung, esophagus, skin, lymph node, brain, ovary, stomach, uterus, testis, and prostate. In one example, the nucleic acid, protein or peptide is used as a vaccine.

A bioactive agent can be introduced into a cell alone or in combination with other therapies known in the art, e.g., chemotherapeutic regimens, bleomycin, radiation, and surgery, to treat various types of proliferative disorders or cancer, or diseases associated with these proliferative disorders or cancers. In addition, the bioactive agent delivered by methods of the invention can be administered in combination with other treatments designed to enhance immune responses, e.g., by co-administration with adjuvants, vitamins, immunostimulatory agents, or cytokines (or nucleic acids encoding cytokines), as is well known in the art. Compositions containing nucleic acids and immunostimulatory agents are described herein.

A bioactive agent introduced into a cell by the methods of the invention can also be used in the manufacture of a medicament for the prevention or treatment of various cancers, or conditions associated with these cancers.

The bioactive agents described herein can also be used in ex vivo treatment. For example, cells such as dendritic cells, peripheral blood mononuclear cells, or bone marrow cells can be obtained from an individual or an appropriate donor and activated ex vivo with a nucleic acid composition, and then returned to the individual. In addition, a nucleic acid expression vector can be introduced into cells such as myoblasts, and then returned to the individual.

The bioactive agents described herein can also be used to modulate the immune response of a mammal with a disease condition that would benefit from the immune modulation. “Modulating the immune response” as used herein is meant to refer to any method of changing the immune response in a mammal that will be beneficial to the treatment of disease conditions. Examples of modulating the immune response include redirecting a mammal's immune response from a Th2 to a Th1 response by inducing monocytic and other cells to produce Th1 cytokines, changing activity of T cell population to prevent symptoms of the condition, inducing proliferation of B cells and increasing immunoglobulin (Ig) secretion.

In Vitro and Ex Vivo Delivery of Microparticles to a Cell

Microparticles containing a bioactive agent such as DNA can be suspended in saline, buffered salt solution, tissue culture medium, or other physiologically acceptable carrier. For in vitrolex vivo use, the suspension of microparticles can be added either to cultured adherent mammalian cells or to a cell suspension. The cells are then subjected to electroporation. Following a 1-24 hour period of incubation, those particles not taken up are removed by aspiration or centrifugation over fetal calf serum. The cells can be either analyzed immediately or recultured for future analysis.

Uptake of microparticles containing a bioactive agent such as DNA into the cells can be detected by PCR, or by assaying for expression of the nucleic acid. For example, one could measure transcription of the nucleic acid with a Northern blot, reverse transcriptase PCR, or RNA mapping. Protein expression can be measured with an appropriate antibody-based assay, or with a functional assay tailored to the function of the polypeptide contained in the microparticle or encoded by the nucleic acid. For example, cells expressing a nucleic acid encoding luciferase can be assayed as follows: after lysis in the appropriate buffer (e.g., cell lysis culture reagent, Promega Corp, Madison Wis.), the lysate is added to a luciferin containing substrate (Promega Corp) and the light output is measured in a luminometer or scintillation counter. Light output is directly proportional to the expression of the luciferase gene.

If the bioactive agent is a nucleic acid that encodes a peptide known to interact with a class I or class II MHC molecule, an antibody specific for that MHC molecule/peptide complex can be used to detect the complex on the cell surface of the cell, using a fluorescence activated cell sorter (FACS). Such antibodies can be made using standard techniques (Murphy et al. Nature, Vol. 338, 1989, pp. 765-767). Following incubation with microparticles containing a nucleic acid encoding the peptide, cells are incubated for 10-120 minutes with the specific antibody in tissue culture medium. Excess antibody is removed by washing the cells in the medium. A fluorescently tagged secondary antibody, which binds to the first antibody, is incubated with the cells. These secondary antibodies are often commercially available, or can be prepared using known methods. Excess secondary antibody must be washed off prior to FACS analysis.

One can also assay by looking at T or B effector cells. For example, T cell proliferation, cytotoxic activity, apoptosis, or cytokine secretion can be measured.

Alternatively, one can directly demonstrate intracellular delivery of the particles by using nucleic acids which are fluorescently labeled, and analyzing the cells by FACS or microscopy. Internalization of the fluorescently labeled nucleic acid causes the cell to fluoresce above background levels. Because it is rapid and quantitative, FACS is especially useful for optimization of the conditions for in vitro or in vivo delivery of nucleic acids. Following such optimization, use of the fluorescent label is discontinued.

If a nucleic acid itself directly affects cellular function, e.g., if it is a ribozyme or an antisense molecule, or is transcribed into one, an appropriate functional assay can be utilized. For example, if the ribozyme or antisense nucleic acid is designed to decrease expression of a particular cellular protein, the expression of that protein can be monitored.

In Vivo Delivery of Microparticles to a Cell

Microparticles containing a bioactive agent such as a nucleic acid can be introduced into a cell of a mammal according to the methods of the invention intramuscularly, topically, intradermally, or subcutaneously. For example, microparticles can be injected intramuscularly followed by electroporation to permit efficient cellular entry of the bioactive macromolecules.

In another example, microparticles can be reconstituted in a paste-forming polymers such as high concentrations of poly(ethylene oxide)-co-poly(propylene oxide)-co-poly(ethylene oxide) (PEO-PPO-PEO) and applied topically to healthy or diseased skin prior to electroporation.

In another example, microparticles can be reconstituted in a polymeric solution that can chemically crosslink into a tissue-adhering (skin) hydrogel, holding the microparticles in place. The applied area can then be electroporated to enhance cellular uptake of the bioactive agent, e.g., a nucleic acid.

Methods of Monitoring Nucleic Acid Activity

The activity of nucleic acids such as a ribozymes, antisense oligonucleotides, or molecules that promote RNA interference can be monitored by analyzing the presence of a targeted RNA or the expression or activity of a protein encoded by a targeted RNA. For example, the activity of an antisense oligonucleotide targeting a VEGF nucleic acid can be monitored by analyzing the amount of VEGF RNA (using, e.g., Taqman RT-PCR analysis), VEGF protein (using, e.g., ELISA or Western analysis), or VEGF activity (measuring blood vessel growth).

The activity of a CpG oligonucleotide can be measured by monitoring the desired effect on an immune response (e.g., tumor reduction, increased lifespan, NK cell activity, inflammation, cytokine release, or T or B cell response to antigen).

The activity of a therapeutic oligonucleotide can be measured by detecting the presence of a desired protein. For example, the activity of a polylC oligonucleotide designed to elicit an interferon response can be determined by measuring serum or tissue levels of interferon.

Methods of Monitoring Gene Expression

Expression of a nucleic acid can be monitored by an appropriate method. For example, expression of a reporter protein can be monitored for example by ELISA, HPLC, mass spectrometry, chemiluminescense, Western, RT-PCR, or immunohistochemistry. Expression of a nucleic acid encoding an immunogenic protein of interest can be assayed by detecting a cytokine, antibody or T cell response to the protein.

Antibody responses can be measured by testing serum in an ELISA assay. In this assay, the protein of interest is coated onto a 96 well plate and serial dilutions of serum from the test subject are pipetted into each well. A secondary, enzyme-linked antibody, such as anti-human, horseradish peroxidase-linked antibody, is then added to the wells. If antibodies to the protein of interest are present in the test subject's serum, they will bind to the protein fixed on the plate, and will in turn be bound by the secondary antibody. A substrate for the enzyme is added to the mixture and a colorimetric change is quantitated in an ELISA plate reader. A positive serum response indicates that the immunogenic protein encoded by the microparticle's DNA was expressed in the test subject, and stimulated an antibody response. Alternatively, an ELISA spot assay can be employed.

T cell proliferation in response to a protein following intracellular delivery of microparticles containing nucleic acid encoding the protein is measured by assaying the T cells present in the spleen, lymph nodes, or peripheral blood lymphocytes of a test animal. The T cells obtained from such a source are incubated with syngeneic APCs in the presence of the protein or peptide of interest. Proliferation of T cells is monitored by uptake of ³H-thymidine, according to standard methods. The amount of radioactivity incorporated into the cells is directly related to the intensity of the proliferative response induced in the test subject by expression of the microparticle-delivered nucleic acid. A positive response indicates that the microparticle containing DNA encoding the protein or peptide was taken up and expressed by APCs in vivo.

The generation of cytotoxic T cells can be demonstrated in a standard ⁵¹Cr release assay. In such an assay, spleen cells or peripheral blood lymphocytes obtained from the test subject are cultured in the presence of syngeneic APCs and either the protein of interest or an epitope derived from this protein. After a period of 4-6 days, the effector cytotoxic T cells are mixed with ⁵¹Cr-labeled target cells expressing an epitope derived from the protein of interest. If the test subject raised a cytotoxic T cell response to the protein or peptide encoded by the nucleic acid contained within the microparticle, the cytotoxic T cells will lyse the targets. Lysed targets will release the radioactive ⁵¹Cr into the medium. Aliquots of the medium are assayed for radioactivity in a scintillation counter.

The generation of cytotoxic T cells can also be demonstrated using an ELISpot assay. A commercially prepared IFN-γ ELISpot kit (R&D Systems, Minneapolis, Minn.) can be utilized per the manufacturer's suggested protocol. Each well of a 96-well hydrophobic polyvinylidene flouride (PVDF) membrane backed plate is pre-absorbed with anti-IFN-γ monoclonal antibody (mAb) and blocked with 10% RPMI for 20 minutes. Approximately 10⁴-10⁵ effectors are then mixed with 10⁵ targets for 18-20 hours at 37° C. in 5% CO₂. Next, each well is washed four times and incubated overnight at 4° C. with a biotinylated non-competing anti-IFN-γ mAb. Wells are then washed three times, incubated for two hours at room temperature with streptavidin alkaline-phosphatase, washed again three times and developed with a 30 minute incubation with BCIP/NBT and washed extensively with distilled water. IFN-γ secreting cells (spots) are enumerated on an automated ELISpot reader system (Carl Zeiss Inc., Thornwood, N.Y.) with KS ELISpot Software 4.2 by Zellnet Consulting, Inc. (New York, N.Y.).

Assays, such as ELISA or FACS, can also be used to measure cytokine profiles of responding T cells.

The use of a plasmid encoding secreted protein permits serum sampling and analysis for expressed protein without sacrificing the animal. Examples of such secreted proteins include secreted embryonic alkaline phosphatase gene, Factor VIII, Factor IX, erythropoietin (EPO), endostatin, aMSH, various cytokines, insulin, and bone morphogenic protein (BMP).

In one example, a plasmid encoding the human secreted embryonic alkaline phosphatase gene (pgWiz™ SEAP, henceforth referred as SEAP) can be used for monitoring systemic expression. SEAP, a secreted form of the membrane bound placental alkaline phosphatase, has a half-life from minutes to a few days in serum. A protein with a short half-life is especially useful to reliably determine expression kinetics.

Levels of enzymatically active SEAP in mouse serum are measured using the Tropix Phospha-Light luminometric assay kit (Applied Biosystems, Foster City, Calif.). Luminescence measurements are performed using a Topcount plate reader (Packard Instruments, Illinois) following 40 minutes of incubation in the reaction buffer. Serum SEAP levels at each time point are expressed in nanograms/ml using the standard curve generated from the positive control (purified human placental alkaline phosphatase) supplied with the assay kit. The data is further analyzed using a Thompson-Tau outlier analysis (Wheeler and Ganji, Introduction to Engineering Experimentation, Prentice Hall, 1996, pages: 142-145) and is plotted as average and standard deviations.

The following are examples of the practice of the invention. They are not to be construed as limiting the scope of the invention in any way.

### EXAMPLES

### Example 1

### High Level Gene Expression Achieved by In Vivo Electroporation of Plasmid DNA-Containing Microparticles

Synthesis of Microparticles

A plasmid encoding secreted alkaline phosphatase (SEAP) was obtained from Aldevron, LLC (Fargo, N. Dak.) and utilized to assess gene expression in vivo. In these experiments, plasmid DNA-containing microparticles comprised of poly(lactide-co-glycolide, random 50:50 L:G) (PLG; Boehringer Ingelheim, Germany) were synthesized and characterized using a modified water/oil/water (w/o/w) emulsion process. 10.6 mg of plasmid DNA and 1.5 mg of polyethylene oxide distearoyl phosphatidyl ethanolamine (PEG-DSPE; Genzyme Corp., MA) were dissolved in 1.6 ml TE pH 8.0 (Tris 10 mM, EDTA, 1 mM)/303 mM sucrose buffer, pH 8.0. The solution was emulsified by homogenization together with 1 g of PLG in 17 ml methylene chloride using a Silverson SL2T mixer with a 16 mm internal diameter homogenization probe (Silverson Machines Inc.; East Longmeadow, Mass.). The addition of the aqueous DNA solution into the organic PLG phase occurred over a period of 20 seconds at ambient temperature. After homogenization of the water/oil emulsion for 4 minutes, an additional 18 ml of methylene chloride was added to the homogenate. Homogenization of the emulsion was performed for an additional 30 seconds.

The emulsion was then homogenized at 6000 RPM for 2.5 minutes with a continuous flow of 1 liter of an aqueous solution containing 1 w/v % PVA and 303 mM sucrose (Poly(vinyl alcohol), molecular weight 23,000 g/mol, Sigma Inc, St Louis, Mo.) using a L4R homogenizer fitted with an in-line mixer. The w/o/w emulsion was stirred at 37° C. for 2.5 hours. The emulsion was centrifuged at 1500 RPM for 15 minutes. The supernatant was discarded, and the pellet suspended in deionized water. The suspension was centrifuged again at 1500 RPM for 15 minutes, supernatant discarded and the pellet resuspended in deionized water. The washed suspension was lyophilized under vacuum (<10 mm Hg) at ambient temperature (˜19-21° C.) for 12 hours to obtain a white, flaky, flocculated powder. Vials containing the lyophile were sealed under nitrogen. The powder was stored at −20° C.

Ultrastructure (Surface Texture, Intactness, Shape)

Scanning Electron Micrographs (SEM) were obtained of the gold-sputtered microparticles using an AMR-1000 scanning electron microscope operated at an accelerating voltage of 10 kV.

Sizing (Volume_{avg}, Number_{avg}

2.5 mg of microparticles were reconstituted in 200 μl of TE buffer, pH 8.0, and examined for appropriate reconstitution. The reconstituted particles were visually examined for aggregation. Sizing of the reconstituted microparticles was carried out on a Coulter Multisizer II (Beckman Coulter, Hialeah, Fla.).

Encapsulation (μg DNA/mg Lyophile)

500 μl of chloroform was added to approximately 2.5 mg of DNA-containing microparticles (weighed out in a 1.5 ml microfuge tube) to dissolve the PLG polymer. 200 μl of TE buffer was added to this solution. The biphasic solution was rotated end-over-end on a LabQuake Rotator (VWR, Chicago, Ill.) at room temperature for 90 minutes to facilitate extraction of DNA into the aqueous phase. 100 μl of the aqueous supernatant was drawn off for analysis. The supernatant was measured at 260 nm by UV spectrophotometry. DNA concentration in the microparticles (μg/mg) was calculated by Beer-Lambert's equation.

DNA Supercoiling (%)

The DNA-containing aqueous extract (described in the previous section) was used to determine supercoiling of DNA in the microparticles by agarose gel electrophoresis. Utilizing encapsulation values determined earlier, a volume corresponding to 250 ng of DNA was loaded onto the ethidium bromide/agarose gel. A qualitative measurement was carried out to determine percent of supercoiled DNA.

Burst DNA (%)

Burst DNA is defined as near-surface DNA released into the saline solution, post reconstitution at room temperature. Approximately 2.5 mg of microparticles were weighed into a 1.5 ml microfuge tube, and reconstituted gently with 0.9% saline at ambient temperature. After 5 minutes (static), the suspension was centrifuged at 3000 rpm for 10 minutes. The supernatant was drawn off and centrifuged a second time at 10,000 RPM for an additional 10 minutes. The supernatant was drawn with a micro-tipped pipette and analyzed by UV spectrophotometry at X=260 nm. Percent DNA released was calculated by Beer-Lambert's equation.

Table 1 summarizes the physico-chemical characterization of PLG microparticles, which had an average DNA encapsulation of 5.6 μg/mg and size distribution of less than 10 μm, by volume (V_{avg}) and number average (N_{avg}) distributions. Encapsulated DNA had high percent supercoiling (˜95%) as determined by agarose gel electrophoresis. Microparticles had a burst of 19% in saline on reconstitution. Microparticles were intact, smooth and spherical, as determined by SEM.

TABLE 1
Physico-Chemical Characteristics of Microparticles

| Appearance (SEM) | Intact, Smooth, Spherical |
|---|---|
| Size (number_{avg}, volume_{avg}) (microns) | 2.1, 5.2 |
| Encapsulation* (μg DNA/mg lyophile) | 5.6 ± 0.35 |
| Supercoiling | 95% |
| Burst (%) | 19.2 |

Mouse Injections

50 μg of plasmid DNA encapsulated in microparticles and encoding human SEAP was suspended in saline and injected into the anterior tibialis muscle of mice (C57BL/6, female, 4-6 weeks old; n=10/group). Saline-injected mice were used as controls. Mice receiving electroporation, were electroporated at the injection site immediately after injection of the microparticles. Electroporation was performed with 2-needle array tips (#533, 0.5 cm gap) by insertion into the muscle so that the array of needles surrounded the injection site using the following conditions: 100 V, 8 pulses, 20 ms pulse length, 1 sec interval between pulses, and unipolar polarity (Genetronics electroporator, ECM 830, BTX Inc., San Diego, Calif.). The electroporation needles were placed into the muscle immediately after injection, on either side of the injection site.

At each time point, mice were bled retro-orbitally, and serum was separated by centrifugation. SEAP levels were measured in serum utilizing a Tropix Phospha-Light luminometry kit according to the manufacturer's instructions (Applied Biosystems, Foster City, Calif.) at pre-determined time points. The data was plotted in SEAP (ng/ml) versus time (days).

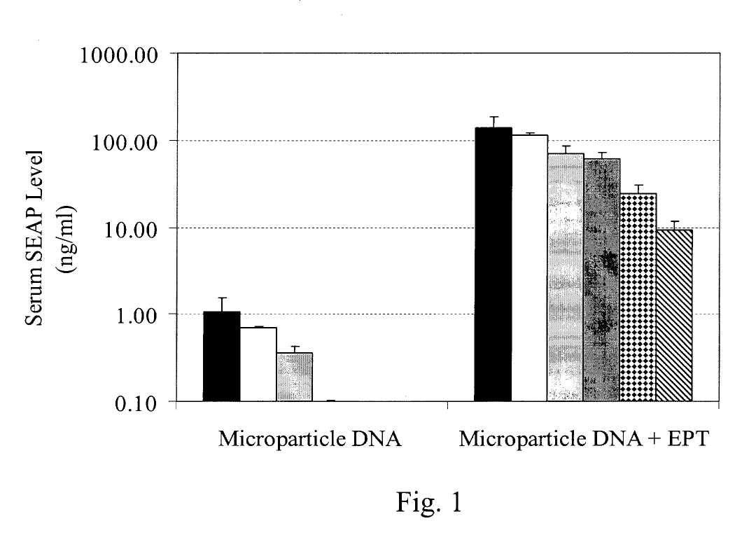

FIG. 1 demonstrates an increase in gene expression obtained when electroporation is applied at the injection site. Sera from saline-injected mice were negative at all time points (<0.3 ng/ml; data not shown). Serum SEAP levels were sustained in mice receiving microparticles with electroporation, even at 300 days post-electroporation, in comparison to mice receiving microparticles with no electroporation (FIG. 1). The incidence of mice expressing SEAP also increased in the presence of electroporation (Table 2). All mice receiving microparticles with electroporation had positive serum SEAP production (defined as greater than 0.3 ng/ml SEAP in serum) up to 300 days following the treatment. In the absence of electroporation, the incidence of mice expressing serum SEAP was 60% positive at day 7 and dropped thereafter.

TABLE 2
Percentage of animals expressing >0.3 ng/ml SEAP

| | Day | Day | Day | Day | | |
|---|---|---|---|---|---|---|
| Group | 7 | 21 | 49 | 90 | Day 200 | Day 300 |
| Microparticle- | 60 | 100 | 40 | 0 | 0 | 0 |
| encapsulated DNA | | | | | | |
| (−electroporation) | | | | | | |
| Microparticle- | 100 | 100 | 100 | 100 | 100 | 100 |
| encapsulated DNA | | | | | | |
| (+electroporation) | | | | | | |

### Example 2

### Enhanced Immune Responses and Gene Expression Achieved by In Vivo Electroporation of DNA-Containing PLG Microparticles: A Dose Response Study

Synthesis and Characterization

Poly(lactide-co-glycolide) (PLG, Boehringer Ingelheim, Germany) microparticles containing either SEAP plasmid (pSEAP) or β-Gal plasmid DNA (pβ-Gal) were synthesized and characterized using the process described above. SEAP plasmid-containing particles had 6.95 μg DNA/mg lyophile and β-Gal plasmid-containing particles contained 3.94 μg DNA/mg lyophile.

Mouse Injections

Microparticles were reconstituted in 0.9% sterile saline to deliver DNA doses of 30, 10, 3 μg/mouse muscle (pβ-gal: 7.61 mg lyophile/30 μg DNA/50 μL of saline, 2.53 mg lyophile/10 μg DNA/50 μl saline, 0.76 mg lyophile/3 μg DNA/50 μl saline; pSEAP: 4.32 mg lyophile/30 μg DNA/50 μL of saline, 1.44 mg lyophile/10 μg DNA/50 μl saline, 0.44 mg lyophile/30 μg DNA/50 μl saline). Balb/c mice (female, 4-6 weeks) received injections of 30 μg, 10 μg or 3 μg of each formulation. SEAP formulations were injected in the right tibialis of each animal and β-gal formulations were injected in the left tibialis muscle of each animal. For mice in groups receiving electroporation, each tibialis muscle was electroporated at the injection site immediately following injection. Electroporation was performed with 2-needle array tips (#533, 0.5 cm gap) by insertion into the muscle so that the array of needles surrounded the injection site using the following conditions: 100 V, 8 pulses, 20 ms pulse length, 1 sec interval between pulses, and unipolar polarity (Genetronics electroporator, ECM 830, BTX Inc., San Diego, Calif.). The electroporation needles were placed into the muscle immediately after injection, on either side of the injection site. Negative control animals received an injection of 50 μL saline per tibialis muscle.

To summarize, groups of mice were treated as follows:

Saline, n=9 (−electroporation)

Test Group: n=5, 30 μg of each encapsulated plasmid DNA/mouse (−electroporation)

Test Group: n=5, 30 μg of each encapsulated plasmid DNA/mouse (+electroporation)

Test Group: n=5, 10 μg of each encapsulated plasmid DNA/mouse (−electroporation)

Test Group: n=5, 10 μg of each encapsulated plasmid DNA/mouse (+electroporation)

Test Group: n=5, 3 μg of each encapsulated plasmid DNA/mouse (−electroporation)

Test Group: n=5, 3 μg of each encapsulated plasmid DNA/mouse (+electroporation)

Serum Collection

Blood was collected from each mouse by retro-orbital bleeding at 7, 23 and 42 days. Blood was allowed to clot and serum was collected by centrifugation at 10,000 rpm for 15 minutes.

SEAP Assays

Enzymatically active SEAP in mouse serum was measured at 7 days, using the Tropix Phospha-Light luminometric assay kit. Assays were performed according to the manufacturer's protocol with the following modifications: 1) SEAP protein samples for the standard curve were prepared in a 1:4 dilution of normal mouse sera; 2) all experimental serum samples were also diluted 1:4 in the manufacturer-supplied dilution buffer; and 3) following 40 minutes of incubation in the reaction buffer, luminescence measurements were analyzed using a commercially available luminescence reader. Serum SEAP levels at each time point were expressed in ng/ml.

β-Gal-Specific Antibody Titers

Sera were collected from mice by retro-orbital bleeding at 23 days post-immunization. Titers of β-gal-specific IgG at 23 days were determined by ELISA. For the analysis of serum antibodies, 96-well plates were incubated at room temperature for 3 hours with β-gal protein at 2 μg/ml in PBS. Plates were blocked for 1.5 hrs with 1% BSA in PBS. Anti-β-gal IgG ELISAs were performed in the following manner: the solid phase was incubated overnight at 4° C. with normal mouse serum (NMS) or antiserum, or β-gal specific monoclonal Ab (Calbiochem Novabiochem, Pasadena, Calif.) followed by incubation with horseradish peroxidase (HRP)-conjugated antibodies specific for mouse IgG (H+L). The binding of antibodies was measured as absorbance at 405 nm after reaction of the immune complexes with ABTS substrate (Zymed, San Francisco, Calif.). Titers were defined as the highest serum dilution that resulted in an absorbance (OD 405) value twice that of non-immune sera at that same dilution.

T cell responses

T cells from pooled splenocytes of immunized or untreated naive mice were purified using enrichment columns (R&D systems, Minneapolis, Minn.) at 42 days post-immunization. Purified CD3+ T cells (2×10⁵) were stimulated with 2×10⁵ irradiated β-gal or HBV peptide pulsed syngeneic spleen cells for 24 hrs. T cell responses were determined by ELISPOT analysis according to the manufacturer's directions (R&D Systems).

Number of Positive Responders

Seven days post injection and at the 30 μg dose, 4/5 mice (80%) injected with encapsulated SEAP DNA expressed SEAP above background levels (0.3 ng/ml) (Table 3). In comparison, electroporation enhanced the number of responders, as SEAP expression was detected in 5/5 mice (100%) at the same dose. For the 10 μg dose, 3/5 mice (60%) injected with SEAP DNA-containing microparticles expressed SEAP greater than 0.3 ng/ml. Electroporation enhanced the number of positive responders to 5/5 mice (100%) at the 10 μg dose. For the 3 μg dose, 1/5 mice (20%) injected with SEAP DNA-containing microparticles expressed SEAP greater than 0.3 ng/ml. In contrast, electroporation enhanced the number of positive responders to 5/5 mice (100%).

TABLE 3
Incidence (%) of Animals Expressing SEAP Increases with
Electroporation

| | | Microparticles + |
|---|---|---|
| | Microparticles (%) | Electroporation (%) |
| DNA Dose (μg) | (# positives/total) | (#positives/total) |
| 3 | 20 | 100 |
| | (1/5) | (5/5) |
| 10 | 60 | 100 |
| | (3/5) | (5/5) |
| 30 | 80 | 100 |
| | (4/5) | (5/5) |

SEAP Expression

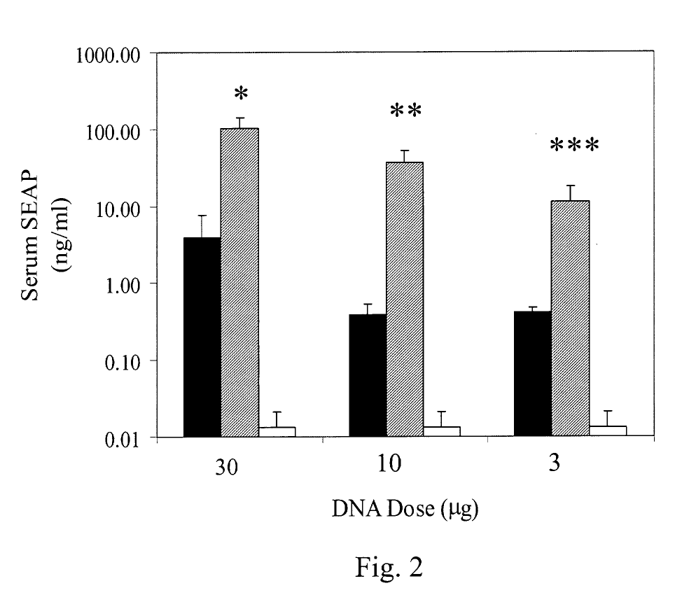

Responding Balb/c mice that received microencapsulated SEAP with electroporation had higher levels of serum SEAP at day 7 than animals which did not receive electroporation (30 μg, 3.9 vs 101 ng SEAP/mL; 10 μg, 0.4 vs 36.7 ng SEAP/ml; 3 μg, 0.4 vs 11.42 ng SEAP/ml) (FIG. 2).

β-Gal Immune Response

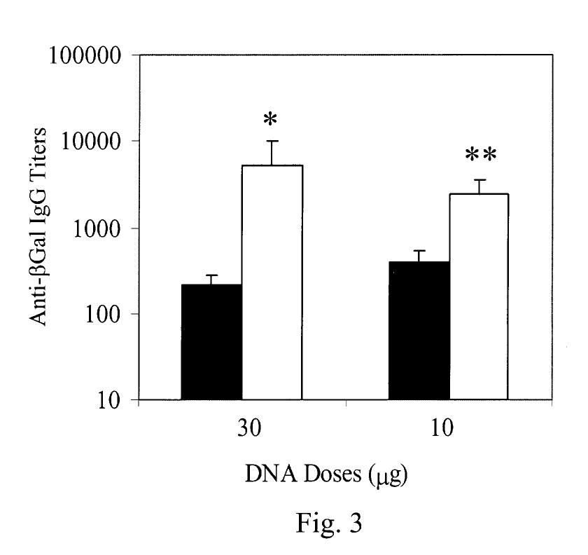

Average titers of anti-β-gal antibodies (IgG) were evaluated at 23 days post immunization. Enhancement of β-gal antibody titers was achieved when electroporation was used in conjunction with microparticle-mediated delivery of β-gal DNA (FIG. 3). Mean anti-β-gal antibody titers were as follows: 30 μg, 217 vs 5250; 10 μg, 390 vs 2462. Positive responses were not detected at this time point in animals receiving 3 μg β-gal DNA, with or without electroporation. The incidence of responding animals also increased when electroporation was used in conjunction with delivery of the particle formulation. For animals receiving 30 μg of encapsulated β-gal the number of responders was 1/5 (without electroporation) and 5/5 (with electroporation). For animals receiving 10 μg of encapsulated 1-gal the number of responders was 1/5 (without electroporation) and 4/5 (with electroporation).

Induction of MHC Class I-Restricted, β-gal₍₈₇₆₋₈₈₄₎ Specific T Cells

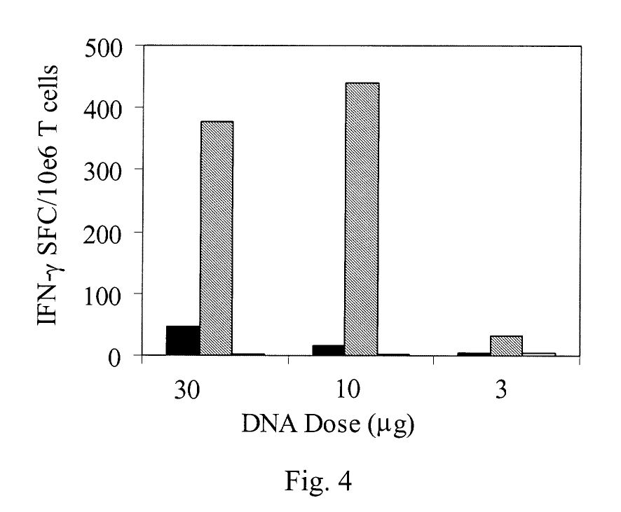

Electroporation enhanced MHC Class I restricted T cell responses at 42 days, at all dose levels as measured by INF-γ ELISPOT (FIG. 4).

### Example 3

### Delivery of Nucleic Acid in P4-AM/P4-SG Networks Combined With Electroporation Enhances the Level of Gene Expression

Materials

Polyethylene oxide-tetraamine (P4-AM; SunBioWest, South Korea), Poly(ethylene oxide)-tetrasuccinimidyl glutarate (P4-SG; SunBioWest, South Korea), Methoxy-polyethylene oxide 2.5K-distearoylphosphatidyl ethanolamine (mPEG-DSPE; Genzyme Corporation, MA), SEAP plasmid DNA (Zycos Inc., MA), and Brookfield Viscometer (cp40 spindle, Brookfield, Inc., Middleboro, Mass.).

Formulations

3% w/v P4-AM/P4-SG was formulated with mPEG-DSPE (10 μg/100 μl) and (100 μg/100 μl) SEAP DNA to create a GT20 formulation. GT20 denotes a gel time of 20 minutes, post reconstitution with buffer at pH 8 as measured by viscometry at 25° C.

Mouse Injections

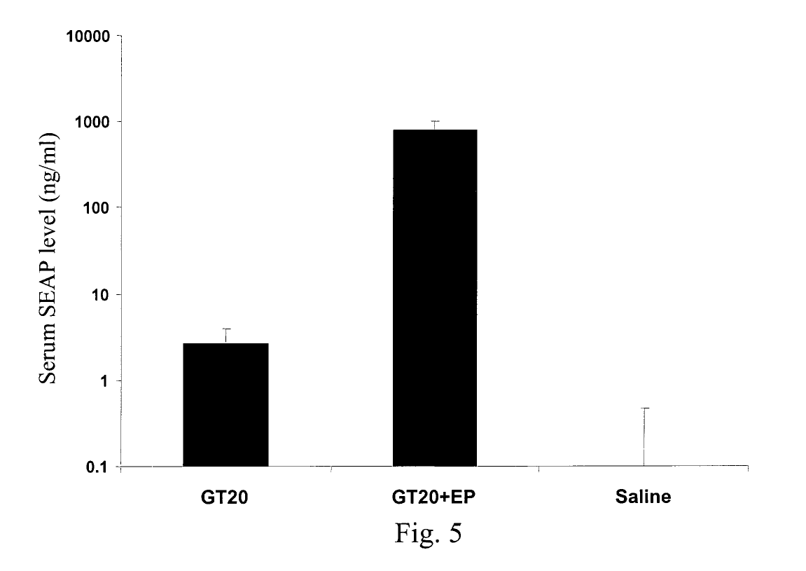

Mice were mildly anesthetized using isofluorane and injected with a 3% w/v P4-AM/P4-SG network formulation or with unformulated plasmid DNA (in saline) bilaterally in the anterior tibialis muscles. All animals were injected with 50 μg of plasmid DNA in an injection volume of 50 μl per muscle. The mouse muscles were electroporated immediately post-injection of the formulations with 2-needle array tips (#533, 0.5 cm gap) by insertion into the muscle so that the array of needles surrounded the injection site using the following conditions: 100 V, 8 pulses, 20 ms pulse length, 1 sec interval between pulses, and unipolar polarity (Genetronics electroporator, ECM 830, BTX Inc., San Diego, Calif.). The electroporation needles were placed into the muscle immediately after injection, on either side of the injection site. Following injection and electroporation, mice were bled retro-orbitally at pre-determined timepoints; serum was collected and analyzed for SEAP as previously indicated in Example 1. Enhancement of SEAP expression was obtained by DNA delivery in network formulation when coupled with electroporation (FIG. 5).

### Example 4

### Electroporation-Combined Delivery of DNA via Microparticles Co-Encapsulated With a Cell-Lytic Peptide, Adjuvant, Bioavailability Enhancer, Lipid or Surfactant

Materials

Plasmid DNA encoding human SEAP (as described in the previous examples) is used as a reporter gene to assess gene expression in vivo. An excipient is co-encapsulated with the plasmid DNA within PLG microparticles. The excipient is any of a cell-lytic peptide (e.g., Mellitin, Magainin I, Streptolysin O; Sigma, Inc. St. Louis, Mo.), a surfactant (e.g., L62; BASF, Inc., Charlotte, Va.), Daan adjuvant (e.g., Monophosphoryl lipid A; Sigma, Inc., St. Louis, Mo.), a bioavailability enhancer (e.g., Vitamin E polyethylene glycol succinate, Eastman Chemical, Inc., Kingsport, Term.), a charged polymer (e.g., poly(amidoamine) (PAMAM); Dendritech, Inc; Midland Mich.), poly(glutamic acid) (Polysciences, Inc., Warrington Pa.), or a charged lipid (e.g., sodium lauryl sulfate, or cetyl trimethylammonium bromide; Sigma, Inc., St. Louis, Mo.).

Formulation

Microparticles are synthesized and characterized as in Example 1. Approximately 0.1 to 50 percent of excipient is dissolved in an aqueous phase containing the plasmid DNA prior to formation of the primary emulsion. Excipients not soluble in water (e.g., Mellitin), are dissolved in the organic methylene chloride phase. Microparticles containing an excipient of choice are characterized for physico-chemical characteristics as described in Example 1.

Mouse Injections

Mice (C57BL/6, female, 4-6 weeks old) are injected intramuscularly one time with microparticles containing 50 μg of pDNA (n=8/group) and the excipient of choice. DNA-containing PLG microparticles with no excipient, and saline injected animals are used as appropriate controls. For the groups receiving electroporation, mouse muscles are electroporated immediately after injection of the microparticles. Electroporation is carried out as described in Example 1. At selected time points, mice are bled retro-orbitally, and serum is separated by centrifugation. Bioactive SEAP levels are measured in serum at pre-determined time points and plotted in nanograms SEAP/ml versus time in days as in FIG. 1.

### Example 5

### Electroporation-Combined Delivery of DNA Via Microparticles Reconstituted in a Solution Containing an Adjuvant, Cell-Lytic Peptide, Charged Polymer, Charged Lipid, Bioavailability Enhancer, or Surfactant

Materials

Plasmid DNA encoding human SEAP is used as a reporter gene to assess gene expression in vivo, as in Example 1. Other reagents are as described in Example 1.

Methods

Microparticles containing the plasmid are synthesized and characterized as in Example 1. The microparticles synthesized herein are reconstituted in an aqueous solution containing 1% w/v of a selected excipient. An excipient dissolved or suspended or emulsified in the aqueous solution is either a cell-lytic peptide (e.g., Magainin I, Streptolysin 0; Sigma, Inc., St. Louis, Mo.), a surfactant (e.g., L62, molecular weight 2000 Da; BASF, Inc., Charlotte, Va.), an adjuvant (e.g., Monophosphoryl lipid A; Sigma, Inc., St. Louis, Mo.), a bioavailability enhancer (e.g., pegylated vitamin E; Eastman Chemical, Inc.), a charged polymer (e.g., poly(amidoamine) (PAMAM); Dendritech, Inc), poly(glutamic acid) (Polysciences, Inc.), a charged lipid (e.g., sodium lauryl sulfate or CTAB; Sigma, Inc., St. Louis, Mo.), or poly(ethylene oxide)-distearoyl phosphatidyl ethanolamine (PEG-DSPE, Genzyme Corp., Cambridge). Microparticles are characterized for physico-chemical characteristics as described in Example 1.

Mouse Injections

Mice (C57BL/6, female, 4-6 weeks old) are injected intramuscularly one time with microparticles containing 50 μg of pDNA (n=8/group) in the excipient of choice. DNA-containing PLG microparticles suspended in the absence of excipient, and saline injected animals are used as appropriate controls. For the groups receiving electroporation, mouse muscles are electroporated immediately after injection of the microparticles. Electroporation is carried out as described in Example 2. At selected time points, mice are bled retro-orbitally, and serum is separated by centrifugation. SEAP assays are performed as described in Example 2.

### Example 6

### Electroporation-Combined Delivery of Microparticle-Encapsulated Oligophosphorothioates

Materials

Oligophosphorothioates (ODN)(m.w. 7500 g/mol, 22-mer; Oligos, Etc., Wilsonville, Oreg.) were encapsulated in microparticles. All other materials and equipment are as described in Example 1.

Encapsulation Process

10.6 mg of oligophosphorothioates was dissolved in 1.6 ml TE pH 8.0 (Tris 10 mM, EDTA, 1 mM)/303 mM sucrose buffer, pH 8.0. The solution was emulsified by homogenization together with 1 g of PLG in 17 ml methylene chloride using a Silverson SL2T mixer with a 16 mm internal diameter homogenization probe (Silverson Machines Inc.; East Longmeadow, Mass.). The addition of the aqueous DNA solution into the organic PLG phase was over a period of 20 seconds at ambient temperature. After homogenization of the water/oil emulsion for 4 minutes, an additional 18 ml of methylene chloride was added to the homogenate. Homogenization of the emulsion was performed for an additional 30 seconds. The emulsion was then homogenized at 6000 RPM for 2.5 minutes with a continuous flow of 1 liter of an aqueous solution containing 1 w/v % PVA and 303 mM sucrose (PVA, Sigma Inc, St Louis, Mo.) using a L4R homogenizer fitted with an in-line mixer. The w/o/w emulsion was stirred at 37° C. for 2.5 hours, then collected by centrifugation (25° C., 10 minutes, 2500 RPM). The supernatant was discarded, and the pellet resuspended in deionized (dI) water. The suspension was centrifuged again at 1500 RPM at room temperature, supernatant discarded and the pellet resuspended in deionized water. The suspension was lyophilized under vacuum (<10 mm Hg) for 12 hours to obtain a white, flocculated powder. Vials containing the lyophile were sealed under nitrogen. The powder was stored at −20° C.

Sizing

2.5 mgs of microparticles were reconstituted in 200 μl of TE buffer, pH 8.0, and examined for aggregation. Sizing of the reconstituted microparticles was carried out on a Coulter Multisizer II (Beckman Coulter).

ODN Encapsulation (μg ODN/mg PLG)

500 μl of chloroform was added to dissolve the polymeric microparticles. The biphasic solution was rotated end-over-end at room temperature for 90 minutes to facilitate extraction of ODN into the aqueous phase. Concentrations of oligophosphorothioates (μg/mg) were determined by HPLC using an anion exchange column (Tosohaas DNA-NPR, UV detection at λ=260 nm). The column was equilibrated overnight at ambient temperature at 0.7 ml/min of mobile phase A. The method included Mobile Phase A comprised of 25 mM NH4Ac (ammonium acetate), 25% ACN (acetonitrile) at pH 8 and Mobile Phase B comprised of 25 mM NH₄Ac, 25% ACN, 500 mM NaOCl₄ (sodium perchlorate), pH 8. To quantitate the concentration of oligonucleotide in the analyte, the following gradient was utilized:

TABLE 4
HPLC Gradient Method

| Time | Flow(ml/min) | % A | % B | % C | % D |
|---|---|---|---|---|---|
| 0 | 0.7 | 100 | 0 | 0 | 0 |
| 3 | 0.7 | 40 | 60 | 0 | 0 |
| 6 | 0.7 | 0 | 100 | 0 | 0 |
| 7 | 0.7 | 0 | 100 | 0 | 0 |
| 10 | 0.7 | 40 | 60 | 0 | 0 |
| 13 | 0.7 | 100 | 0 | 0 | 0 |

A standard curve of 0, 5, 10, 25, 50 and 100 μg/ml of oligonucleotide was constructed and the % RSD calculated on a linear fit regression.

Burst

Percent burst was calculated based on a standard curve generated with 0, 10, 50, 100 μg/ml of oligophosphorothiates in phosphate buffered saline. Burst DNA is defined as near-surface ODN released into the saline solution, post reconstitution at room temperature. 2.5 mgs of microparticles were weighed into microfuge tubes (n=2). 1 ml of saline was added to the microparticles, and the particles suspended gently. The suspension was rotated end-over-end at room temperature for 5 minutes, at the end of which the sample tubes were microcentrifuged for 10 minutes at 3000 RPM. 800 μl of supernatant was drawn from each sample. The burst samples were concentrated by lyophilization of 800 μl of the supernatant. The lyophilized powder was reconstituted in 100 μl of milliQ water, and prepped for HPLC analysis. Oligophosphorothioate concentrations (μg) were determined by High Performance Chromatography, using an anionic exchange column, using the method described previously.

In-vitro Release 2.5 mgs of microparticles (n=3) for timepoints 1 hour, 1 day, 2 days, 3 days, 4 days, 5 days, 6 days, etc. were weighed into 2 ml round bottomed centrifuge tubes and reconstituted with 1 ml of Dulbecco's phosphate buffered saline (PBS)/0.5 mM EDTA, pH 7.0. The tubes were rotated end-over-end in a 37° C. incubator. 800 μl of supernatant was retrieved at each timepoint and replaced with 800 μl of fresh PBS. The in-vitro release samples were concentrated by lyophilization of 800 μl of the supernatant. The lyophilized powder was reconstituted in 100 μl of milliQ water, and prepped for HPLC analysis.

Chemical Compatibility

Compatibility of the formulation with the oligonucleotide was determined by extraction of the ODN from the microparticles (see encapsulation extraction method) followed by HPLC analysis of the extract. To determine if intact ODN was released from the microparticles over time, the retention times of in vitro released ODN was compared with unformulated ODN, over time in days.

Ultrastructure

Scanning Electron Micrographs (SEM) were obtained of the gold-sputtered microparticles (with, and without excipient) using an AMR-1000 scanning electron microscope operated at an accelerating voltage of 10 kV.

Results

The physical appearance of the microparticles was smooth and spherical (by Scanning Electron Microscopy). Table 5 summarizes the physico-chemical characteristics of three batches of microparticles containing oligophosphorothioates. Encapsulations were between 5-8 μg/mg PLG, measured by HPLC. Bursts were <20% measured by HPLC.

TABLE 5
Characterization of Microparticles Containing Oligonucleotides

| | | | Size |
|---|---|---|---|
| | Encapsulation | | (number_{avg}, |
| PLG | (μg ODN/mg | Burst | volume_{age}) |
| Microparticles | lyophile) | (%) | microns |
| Batch 1 | 5.87 | 15.1 | 2.2, 5.6 |
| Batch 2 | 7.51 | 6.89 | 1.9, 5.1 |
| Batch 3 | 8.46 | 11.29 | 2.1, 5.5 |

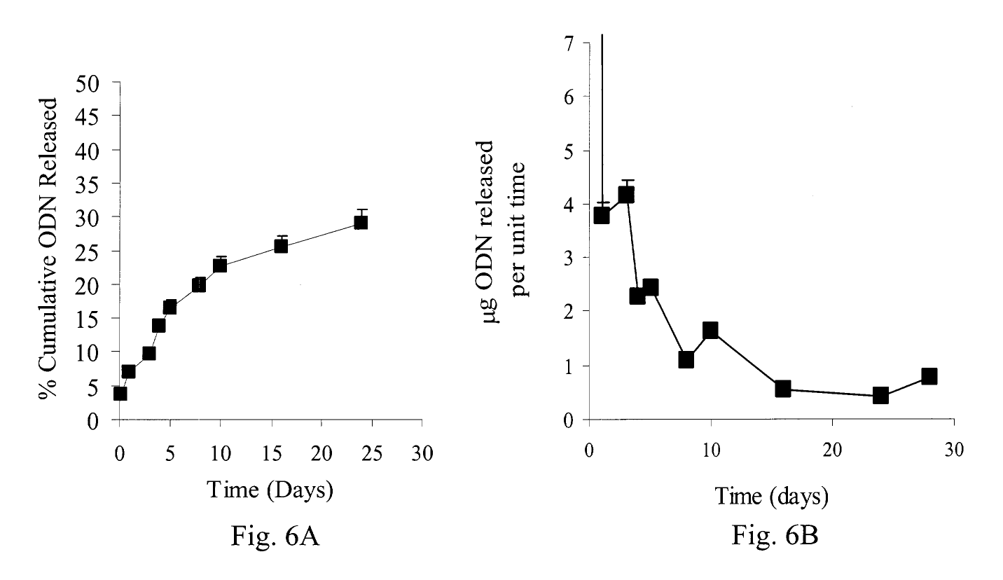

HPLC analysis of ODN extracted from microparticles showed a single peak of oligonucleotide, indicating the microparticle formulation process did not damage the drug (data nor shown). Furthermore, the retention times of released ODN over a period of 46 days was equivalent to unformulated ODN, indicating that intact ODN was released from the microparticles (data not shown). In vitro release of ODN from PLG microparticles indicated 35% cumulative released from PLG microparticles in 25 days (FIG. 6A). FIG. 6B is a plot of micrograms of ODN released per day, over a period of 24 days.

Mouse Immunizations

Microparticles containing encapsulated ODN (e.g., CPG oligophosphorothioates) (−electroporation) are suspended in saline containing hepatitis B surface antigen and are injected subcutaneously into Balb/c mice (n=6/group) at a dose of 100 μg ODN/mouse and 1 μg hepatitis B surface antigen/mouse. Encapsulated ODN and hepatitis surface antigen suspended in sterile saline are injected subcutaneously in Balb/c mice, followed by electroporation. Electroporation is carried out as indicated in Example 1. Saline injected mice (−electroporation) are used as controls. Mice are bled retro-orbitally at various time points post injection and hepatitis B surface antigen IgG, IgG2a levels measured by ELISA.

In another experiment, microparticles containing antisense oligophosphorothioates are suspended in saline and are injected subcutaneously with or without electroporation into Balb/c mice at a daily dose of 0.1-10 μg/kg/mouse. Mice are bled retro-orbitally at various time points post injection. Bioactivity of the antisense oligophosphorothioates is measured by assaying (e.g., by ELISA) for down-regulation of a protein encoded by a mRNA to which the antisense oligophosphorothioate binds.

### Example 7

### Electroporation-Combined Delivery of Microparticle-Encapsulated Nucleic Acids in a Solution Containing a Temperature Sensitive Polymer

Formulation

Microparticles containing a nucleic acid (e.g., plasmid DNA or oligophosphorothioates) are generated and characterized using the methods described in previous examples (Example 1 and 2). A 30% w/v solution of a temperature sensitive polymer such as Pluronic F127® (Poly(ethylene oxide)-co-poly(propylene oxide)-co-poly(ethylene oxide), mw 12000 daltons; (BASF, Inc., Charlotte, Va.) is prepared in phosphate buffered saline, pH 7.4. The solution is maintained at 4° C. Microparticles (e.g., about 50 mg) are reconstituted in 1 ml of the Pluronic solution.

Methods

In an example of topical administration, a solution containing the microparticles is sprayed cold onto skin. The Pluronic-containing solution undergoes a sol-to-gel transition when the solution comes in contact with the skin (at 37° C.). In addition to topical administration, a Pluronic-containing solution can also be injected cold to form a gel depot containing microparticles. Irrespective of the mode of administration, the site of microparticle application is electroporated with multiple pulses. Electroporation is performed with a device designed for dermal EPT such as the DermPulser® Electroporator Device (Genetronics, Inc. San Diego, Calif.).

### Example 8

### Electroporation-Combined Delivery of Microparticle-Encapsulated Nucleic Acids in a Hydrogel

Microparticles containing a nucleic acid (e.g., plasmid DNA or oligophosphorothioates) are generated and characterized using the methods described in previous examples. The microparticles (e.g., about 50 mg) are reconstituted in 1 ml of 10% w/v P4-SH (SunBio Systems, Korea) in phosphate buffer, pH 7.0. The 1 ml solution is then mixed with 1 ml of 10% w/v P4-SG (SunBio Systems, Korea) in 0.1M phosphate buffer, pH 7.5. The microparticle-containing solution (P4-SH+P4-SG) is drawn up into a syringe and applied to the area of application (e.g., tumor resections or intra-tumor resections). Subsequently, the area of application is subjected to electroporation as described herein.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

What is claimed is:

1. A method of introducing a bioactive agent into a living cell, the method comprising:
contacting a living cell with a delivery vehicle comprising a bioactive agent; and
applying an electrical field via electroporation to the cell, under conditions and for sufficient time to allow uptake of the bioactive agent into the cell,
wherein the delivery vehicle is a microparticle or a hydrogel, and wherein the microparticle is not encapsulated in a liposome.

2. The method of claim 1, wherein the bioactive agent is a nucleic acid.

3. The method of claim 2, wherein the nucleic acid is an oligonucleotide.

4. The method of claim 2, wherein the nucleic acid is a plasmid DNA.

5. The method of claim 2, wherein the nucleic acid encodes a polypeptide and the method results in production of the polypeptide by the cell.

6. The method of claim 5, wherein the method results in detectable expression of the polypeptide produced by the cell for a period of at least four weeks.

7. The method of claim 6, wherein the method results in detectable expression of the polypeptide produced by the cell for a period of at least twelve weeks.

8. The method of claim 7, wherein the method comprises detecting expression of the polypeptide produced by the cell after a period of at least twelve weeks.

9. The method of claim 1, wherein the bioactive agent is a peptide nucleic acid.

10. The method of claim 1, wherein the bioactive agent is a polypeptide.

11. The method of claim 1, wherein the contacting and applying steps are carried out on the cell in vitro.

12. The method of claim 1, wherein the cell is contained in a living animal and the method comprises applying an electrode to a tissue of the animal.

13. The method of claim 12, wherein the tissue is a muscle tissue.

14. The method of claim 12, wherein the nucleic acid encodes a polypeptide and the method results in production of the polypeptide by the cell.

15. The method of claim 14, wherein the method results in detectable expression of the polypeptide produced by the cell for a period of at least four weeks.

16. The method of claim 14, wherein the method results in detectable expression of the polypeptide produced by the cell for a period of at least twelve weeks.

17. The method of claim 16, wherein the method comprises detecting expression of the polypeptide produced by the cell after a period of at least twelve weeks.

18. The method of claim 14, wherein the method results in the generation of an immune response within the animal directed against the polypeptide.

19. The method of claim 18, wherein the immune response is a therapeutic immune response.

20. The method of claim 18, wherein the immune response is a prophylactic immune response.

21. The method of claim 12, wherein the method comprises injecting an aqueous solution comprising the delivery vehicle and the bioactive agent into the tissue of the animal.

22. The method of claim 21, wherein the tissue is a muscle tissue.

23. The method of claim 1, wherein the delivery vehicle is a microparticle.

24. The method of claim 23, wherein the microparticle comprises a synthetic polymer.

25. The method of claim 24, wherein the synthetic polymer comprises poly-lactide-co-glycolide.

26. The method of claim 23, wherein the microparticle has biodegradable linkages comprised of lactates, glycolates, lactate-co-glycolates, caproates, trimethylene carbonates or combinations thereof.

27. The method of claim 23, wherein the microparticle is less than 10 μm in diameter.

28. The method of claim 27, wherein the microparticle is at least 1 μm in diameter.

29. The method of claim 23, wherein the microparticle does not comprise a catioinic lipid.

30. The method of claim 1, wherein the delivery vehicle is in an aqueous solution.

31. The method of claim 29, wherein the aqueous solution comprises an excipient.

32. The method of claim 31, wherein the excipient is a cell-lytic peptide, polymer, lipid, adjuvant, or bioavailability enhancer.

33. The method of claim 1, wherein the delivery vehicle is a hydrogel.
